(19) United States
(12) Patent Application Publication
(10) Pub. No.: US 2026/0185062 A1
(43) Pub. Date: Jul. 2, 2026

(54) DNASE 1-LIKE 3 ENGINEERED FOR IMPROVED EXPRESSION AND USE IN THERAPY

(71) Applicant: Neutrolis, Inc., Cambridge, MA (US)

(72) Inventor: Tobias A. FOX, Cambridge, MA (US)

(21) Appl. No.: 19/129,105

(22) PCT Filed: Nov. 14, 2023
(86) PCT No.: PCT/US2023/079602

§ 371 Date: May 12, 2025

Related U.S. Application Data
(60) Provisional application No. 63/425,106, filed on Nov. 14, 2022.

(51) Int. Cl. C12N 9/22; A61K 38/00

(52) U.S. Cl. CPC C12N 9/22; C12Y 301/21001; A61K 38/00; C07K 2319/02; C07K 2319/31; C07K 2319/50

(57) ABSTRACT

The present disclosure provides methods for engineering proteins (e.g., DNase enzymes) for improved production, structural homogeneity, and/or reduced immunity.

### PRIORITY

The present application claims the benefit of, and priority to, U.S. provisional application No. 63/425,106, filed Nov. 14, 2022, the contents of which are hereby incorporated by reference in its entirety.

### FIELD OF THE INVENTION

The present disclosure provides, in part, DNase enzymes engineered for improved production, structural homogeneity, and/or reduced immunogenicity for use in therapy.

### DESCRIPTION OF THE XML FILE SUBMITTED ELECTRONICALLY

This application contains a sequence listing. The sequence listing has been submitted electronically via EFS-Web as an XML entitled “NTR-015PC_119604-5015_sequence_listing.xml.” The sequence listing is 85,383 bytes in size, and was created on Nov. 13, 2023. The sequence listing is hereby incorporated by reference in its entirety.

### BACKGROUND

Enzyme replacement therapy is a promising approach for treatment of various diseases and disorders. For example, inflammatory diseases such as systemic lupus erythematosus (SLE) might be treated with DNase enzymes. Lauková et al., Deoxyribonucleases and Their Applications in Biomedicine, Biomolecules 2020; 10(7): 1036. Enzymes are produced in microbial hosts such as Pichia pastoris or mammalian cells. However, not every protein of interest is produced in or secreted by the desired expression system, such as Pichia pastoris, to high enough titers, among other hurdles. For example, several studies have revealed unwanted secondary modifications, structural heterogeneity, and inefficient processing of the N-terminal signal sequence. See, e.g., Arbeitman et al., Structural and functional comparison of SARS-CoV-2-spike receptor binding domain produced in Pichia pastoris and mammalian cells. Sci Rep 2020; 10:21779 (2020); Reverter et al., Overexpression of Human Procarboxypeptidase A2 in Pichia pastoris and Detailed Characterization of Its Activation Pathway, Protein Chemistry and Structure| 1998; 273(6): 3535-3541; Katla et al., Novel glycosylated human interferon alpha 2b expressed in glycoengineered Pichia pastoris and its biological activity: N-linked glycoengineering approach, Enzyme and Microbial Technology 2019; 128:49-58, Therefore, there is a need for advances in expression systems to allow for production of recombinant therapeutic proteins with improved production titers, structural homogeneity, and/or reduced immunity.

### SUMMARY OF THE DISCLOSURE

In various aspects, the present disclosure provides recombinant proteins that are produced by secretion from expression systems with complete processing of an N-terminal signal sequence. In various aspects, the disclosure provides methods for making such proteins, including DNase enzymes, and for their use in therapy.

In various aspects and embodiments, the present disclosure is based, in part, on the discovery that addition of a linker of four or more amino acids between a secretion signal and a protein of interest can improve its processing and expression titers from microbial expression systems, as well as control post-translational modification profile. The present disclosure is also based, in part, on the discovery that modification of the C-terminus can improve product homogeneity profile of a protein produced in an expression system.

In some aspects, the present disclosure provides a variant of DNASE1-LIKE 3 (D1L3 variant) comprising an N-terminal extension. In some embodiments, the N-terminal extension comprises at least four amino acids. In some embodiments, the N-terminal extension is not subject to post-translational modification by the host cell and/or is non-immunogenic upon administration to a human or animal subject. In some embodiments, the D1L3 variant produced by the host cell comprises a DIL3 enzyme lacking a signal peptide and comprises an amino acid sequence that has at least 80% sequence identity to amino acids 21 to 282 of SEQ ID NO: 4 (Isoform 1) or amino acids 21 to 252 of SEQ ID NO: 5 (Isoform 2).

In some embodiments, the DIL3 variant is produced in a host cell (such as but not limited to Pichia pastoris) by cleavage of an N-terminal signal peptide. Any signal peptide that allows for secretion of the D1L3 in the desired expression system may be used. In some embodiments, the signal peptide is the alpha-mating factor (αMF) pre-pro secretion leader from Saccharomyces cerevisiae (SEQ ID NO: 38).

In various embodiments, the N-terminal extension has a length in the range of 4 to about 18 amino acids. In embodiments, the first N-terminal amino acid is not a Met residue and is not a Gly residue (which as disclosed herein can be subject to post-translational modification). In some embodiments, the last amino acid residue of the N-terminal extension is not a Ser residue, which as disclosed herein can increase immunogenicity risk for DIL3. In some embodiments, the last amino acid residue of the N-terminal extension is not a polar or charged amino acid residue, such as an amino acid residue selected from Ser, Thr, Gln, Asn, Glu, Asp, Arg, His and Lys. In some embodiments, the last amino acid residue of the N-terminal extension is a Gly residue. In some embodiments, the first amino acid residue of the N-terminal extension not a Gly residue, and the last amino acid residue of the N-terminal extension is a Gly residue. In some embodiments, the N-terminal extension is predominately Gly residues (i.e., more than 50% Gly residues). In some embodiments, the linker comprises at least one Cysteine residue. For example, Cysteine can be placed at the N-terminus to provide for site-specific chemical conjugation, such as to polyethylene glycol, or disulfide dimerization. In some embodiments, the N-terminal extension comprises or consists of the amino acid sequence of SGGGG (SEQ ID NO: 60). In some embodiments, the N-terminal extension comprises or consists of the amino acid sequence of CGGGG (SEQ ID NO: 74). In some embodiments, the N-terminal extension comprises or consists of the amino acid sequence SGGSGGSGG (SEQ ID NO: 61). In some embodiments, the N-terminal extension comprises or consists of the amino acid sequence of SGGSGGSGGSGGSGGSGG (SEQ ID NO: 62).

In some embodiments, the N-terminal extension improves expression titer of the D1L3 variant compared to the same DIL3 sequence lacking the N-terminal extension, and with regard to a selected signal sequence and expression host. In some embodiments, the signal peptide is completely removed from the DIL3 variant upon secretion from the host.

In some embodiments, the DIL3 variant further comprises a C-terminal extension reducing heterogeneity. In some embodiments, the C-terminal extension is at least two, or at least three, or at least four, or at least five amino acids in length. In some embodiments, the extension is a hydrophilic sequence of 2, 3, or 4 amino acids. In some embodiments, the C-terminal amino acid is a lysine (Lys) or an arginine (Arg). In some embodiments, the C-terminal extension comprises or consists of the amino acid sequence SSR, which can be employed in some embodiments for D1L3 variants having whole or partial deletions of the C-terminal basic domain (as described further herein). In some embodiments, the DIL3 variant comprises the C-terminal basic domain having the amino acid sequence SSRAFTNSKKSVTLRKKTKSKRS (SEQ ID NO: 75), or a modified version thereof. When these D1L3 enzymes are expressed in Pichia pastoris the resulting polypeptide will be cleaved after the SSR sequence at the beginning of the basic domain sequence. In still other embodiments, the D1L3 is encoded and expressed with a 20 amino acid deletion of the basic domain, thereby having the sequence SSR at the C-terminus.

In some embodiments, the DIL3 variant comprises a deletion of at least three, or at least five, or at least eight, or at least ten, or at least twelve, or at least fifteen, or at least eighteen, or at least twenty, or all 23 amino acids of a C-terminal basic domain (BD) defined by amino acids 283 to 305 of SEQ ID NO: 4. In some embodiments, the DIL3 variant comprises a deletion of 20 amino acids of the C-terminal basic domain (BD) defined by amino acids 283 to 305 of SEQ ID NO: 4, such that the DIL3 variant comprises the C-terminal extension of the sequence SSR.

In various embodiments, the DIL3 variant comprises an amino acid sequence that has at least 70% sequence identity to D1L3 Isoform 1 (SEQ ID NO: 4) or DIL3 Isoform 2 (SEQ ID NO: 5) lacking the BD (i.e., at least 80% sequence identity with amino acids 21 to 282 of SEQ ID NO: 4, or amino acids 21 to 252 of SEQ ID NO: 5), and wherein the DIL3 variant has a deletion of one or more amino acids from the BD. In embodiments, the DIL3 variant comprises the N-terminal extension of any of the embodiments disclosed herein and/or the D1L3 variant comprises the C-terminal extension of any of the embodiments disclosed here.

In some embodiments, the DIL3 variant has a substitution of the amino acid corresponding to C48 of SEQ ID NO: 4. These variants can remove an unpaired cysteine that improves recombinant production and stability of the enzyme.

In some embodiments, the D1L3 variant comprises one or more unpaired cysteines which are configured for and/or capable of dimerization, for example, an unpaired cysteine at position 48 relative to the amino acid sequence of SEQ ID NO: 4 (numbered without the signal peptide). In some embodiments, the disclosure provides a DIL3 dimer according to this disclosure that is dimerized through disulfide bridge at C48. In some embodiments, the D1L3 variant comprises the incorporation of non-native cysteines, which can contribute to dimerization, including incorporation of non-native cysteines at the N-terminus. In such embodiments, the disclosure provides a D1L3 dimer, which can be used in therapy according to the disclosure. In some embodiments, the DIL3 variant comprises a substitution of cysteine at position 48 (C48) with respect to SEQ ID NO: 4, to further control dimerization positions. For example, the mutation can be selected from C48A, C48G, and C48S. In some embodiments, the substitution of C48 (e.g., to C48A or C48G) increases the enzymatic capacity (such as chromatin degradation) of the DIL3 enzyme. In some embodiments, the D1L3 variant has a C-terminal extension which comprises or consists of the amino acid sequence SSR and a mutation of C48A with respect to SEQ ID NO: 4 (numbered without the signal peptide).

Exemplary D1L3 variants in accordance with this disclosure include SEQ ID NO: 63, SEQ ID NO: 66, SEQ ID NO: 70, SEQ ID NO: 71, SEQ ID NO: 72, SEQ ID NO: 73, or SEQ ID NO: 77, where the signal peptide is not present and is completely processed by the host expression system.

In some embodiments, the D1L3 variant comprises a fusion or conjugation to a half-life extending moiety. In some embodiments, the polymer is a polyethylene glycol (PEG). In some embodiments, the PEG is conjugated to the N-terminus. In some embodiments, the PEG polymer connects two DIL3 variant molecules through their N-termini. In some embodiments, the PEG is conjugated to the C-terminus. In some embodiments, the PEG polymer connects two D1L3 variant molecules through their C-termini.

In some embodiments, the D1L3 variant comprises a fusion or conjugation to a half-life extending moiety. In some embodiments, the half-life extending moiety is a fusion partner. In some embodiments, the fusion partner is selected from albumin, transferrin, an Fc, or elastin-like protein, XTEN sequence, or a variant thereof. In some embodiments, the fusion partner is an albumin. In some embodiments, the fusion partner is fused at the N-terminus to mature D1L3 enzyme lacking a signal peptide (and through a linker sequence). In some embodiments, the D1L3 variant comprises the amino acid sequence of SEQ ID NO: 76 or SEQ ID NO: 78, or a variant thereof having at least about 99% sequence identity thereto. In some embodiments, the N-terminal extension of any of the embodiments disclosed herein links the fusion partner with the N-terminus of mature D1L3 enzyme. In some embodiments, the linker is from about 5 to about 50 amino acids, or from about 10 to about 35 amino acids, or from about 15 to about 35 amino acids in length. In some embodiments, the linker comprises the amino acid sequence S(GGS)₄GSS (SEQ ID NO: 23), S(GGS)₉GSS (SEQ ID NO: 24), and (GGS)₉GS (SEQ ID NO: 25).

In some aspects, the present disclosure provides a method for expressing the DIL3 variant of any one of the embodiments disclosed herein. In some embodiments, the method comprises introducing a genetic construct encoding the DIL3 variant of any of the embodiments disclosed herein and comprising a signal peptide in a yeast cell, and recovering the DIL3 variant. In some embodiments, the yeast cell is Pichia pastoris. In some embodiments, the signal peptide is the alpha-mating factor (αMF) pre-pro secretion leader from Saccharomyces cerevisiae (SEQ ID NO: 38). In some embodiments, the fusion protein is synthesized with an N-terminal signal peptide. The signal peptide may be completely removed during secretion from the host cell. With respect to expression in Pichia pastoris, the alpha-mating factor (MF) pre-pro secretion leader from Saccharomyces cerevisiae (SEQ ID NO: 38) may be used for expression. These elements are cleaved during expression, and are not present in the DIL3 variant enzyme product.

In some aspects, the present disclosure provides an isolated polynucleotide encoding the D1L3 variant of any of the embodiments disclosed herein, and which provide advantages for expression ex vivo or in vivo. In some aspects, the present disclosure provides a polynucleotide that is an mRNA or a modified mRNA (mmRNA). In some aspects, the present disclosure provides a polynucleotide that is DNA.

The disclosure in some aspects provides pharmaceutical compositions comprising the D1L3 enzyme described herein, or a polynucleotide encoding the DIL3 enzyme, or a transfection or expression vector comprising the same, or a cell comprising the polynucleotide or vector, and a pharmaceutically acceptable carrier.

In some aspects, the present disclosure provides a pharmaceutical composition comprising an effective amount of the D1L3 variant of any of the embodiments disclosed herein, the DIL3 variant produced according the method of any of the embodiments disclosed herein, the polynucleotide according to any of the embodiments disclosed herein, the vector according to any of the embodiments disclosed herein, or the host cell according to any of the embodiments disclosed herein, and a pharmaceutically acceptable carrier.

In some embodiments, the composition comprises the DIL3 variant of any of the embodiments disclosed herein, and a pharmaceutically acceptable carrier for parenteral administration. In some embodiments, the pharmaceutical composition is formulated for topical, parenteral, or pulmonary administration. In some embodiments, the pharmaceutical composition is formulated for intradermal, intramuscular, intraperitoneal, intraarticular, intravenous, subcutaneous, intraarterial, ocular, oral, sublingual, pulmonary, or transdermal administration.

In embodiments, the method for recombinant production of variants of D1L3 enzyme employs a non-mammalian expression system, e.g., a eukaryotic non-mammalian expression system, such as Pichia pastoris. In embodiments, the Pichia pastoris encodes the DNase enzyme with its native signal peptide allowing for secretion from host cells. In embodiments, the expression system is a mammalian cell expression system, such as Chinese Hamster Ovary (CHO) cells. In embodiments, the method for recombinant production of variants of D1L3 enzyme further comprises isolating and/or purifying the DIL3 enzyme and subjecting the isolated and/or purified the DIL3 enzyme to a modification. In embodiments, the modification comprises conjugation of the isolated and/or purified the D1L3 enzyme to a polymer (without limitation, e.g., PEG). In embodiments, the polymer is added to a specific site using the desired conjugation chemistry (without limitation, e.g., maleimide chemistry).

In other aspects, the present disclosure provides a method for treating a subject in need of extracellular chromatin degradation, extracellular trap (ET) degradation and/or neutrophil extracellular trap (NET) degradation. The method comprises administering a therapeutically effective amount of the DIL3 enzyme or composition described herein.

In some aspects, the present disclosure provides an expression construct for improving processing of a polypeptide precursor in a host, the expression construct comprising a signal peptide fused to the polypeptide via a linker. In some embodiments, the linker has at least three amino acids length. In some embodiments, the signal peptide is completely removed from the polypeptide. In some embodiments, the signal peptide is not removed from the polypeptide. In some embodiments, the signal peptide is incompletely processed in the host in the absence of the linker. In some embodiments, the polypeptide is or comprises an enzyme, a cytokine, a cytokine agonist, a cytokine antagonist, a hormone, a hormone agonist, a hormone antagonist, an antibody or an antigen binding fragment thereof, an antibody-like molecule or an antigen binding fragment thereof, antigen, a component of a vaccine, a fusion protein or a combination thereof.

Other aspects and embodiments of the disclosure will be apparent from the following detailed description and working examples.

### BRIEF DESCRIPTION OF THE DRAWINGS

FIG. 1A and FIG. 1B illustrate the expression of DIL3 in Pichia pastoris using either the native secretion signal or α-mating factor from Saccharomyces cerevisiae (αMF). FIG. 1A shows the N-terminus of D1L3 that was led by the aMF secretion leader from Saccharomyces cerevisiae. FIG. 1B shows that the secretion signal from αMF resulted in glycosylation and non-processing of the signal.

FIG. 2 shows a structural model of a N-terminal native secretion signal from alpha-mating factor linked via a linker to a D1L3 enzyme. Without being bound by theory, it is thought that the flexible linker positions the cleavage site of the alpha-mating factor to enable efficient processing.

FIG. 3 shows mass spectroscopic analysis of an DIL3 enzyme variant comprising BDD_D1L3 enzyme (S283_S305del) produced by the construct comprising Alpha Mating Factor+a GGGGS linker (SEQ ID NO: 58). These data indicated that the secretion signal was properly removed, but the protein contained a post-translational modification at the N-terminal glycine residue (probably myristoylation).

FIG. 4 shows the results of a titration experiment performed to compare the chromatin-degrading activities of the DIL3 variant of SEQ ID NO: 63 and DNase 1 (D1, SEQ ID NO: 1).

FIG. 5A illustrates the structural heterogeneity of the D1L3 variant of SEQ ID NO: 63 as illustrated by mass spectrometry. FIG. 5B illustrates the structural homogeneity of the D1L3 variant of SEQ ID NO: 66 as illustrated by mass spectrometry.

FIG. 6 shows a Western Blot with an anti-DNASE1L3 antibody of culture supernatants of hosts expressing four different DNASE1L3 variants. In all variants, C48 is mutated to alanine or serine. The sample 1 (SEQ ID NO: 70), which was produced by the construct comprising Alpha Mating Factor+a SGGGG linker (SEQ ID NO: 60), was a D1L3 variant having a C48A substitution and a C-terminal extension having the sequence SSR. Samples 2-4 (SEQ ID NOs: 71 to 73, respectively), which were produced by the construct comprising Alpha Mating Factor+CGGGG linker (SEQ ID NO: 74), are DIL3 variants having a C-terminal extension of the sequence SSR, or the wild-type C-terminal basic domain SSRAFTNSKKSVTLRKKTKSKRS (SEQ ID NO: 75). Dimers were detected in DNASE1L3 variants containing an N-terminal cysteine.

FIG. 7 shows a Western Blot analysis of two DNASE1L3 variants that feature a wild-type C-terminal amino acid sequence, i.e., SSRAFTNSKKSVTLRKKTKSKRS (SEQ ID NO: 75), or the modified C-terminal amino acid sequence, i.e., SSR, respectively. The variants were expressed in Pichia pastoris using the alpha-mating factor as a signal sequence in conjunction with the N-terminal SGGGG linker (SEQ ID NO: 60). Western Blot analysis of supernatants did not detect the theoretical mass difference of 2.3 kDa between both variants.

FIG. 8 shows high-molecular weight (HMW)-chromatin degradation assay comparing wild-type D1L3 to C48 variants. Wild-type DIL3 contains an unpaired cysteine in position 48 (e.g., C48). The impact of amino acid substitutions at C48 on enzymatic activity was characterized using HMW-chromatin (i.e., purified nuclei from HEK293 cells) incubated with equal amounts D1L3 variants. Following incubation, DNA was isolated and its degradation was visualized via agarose gel electrophoresis (AGE). Mutation of C48 to C48A or C48G was associated with an increase in enzymatic activity.

FIG. 9 shows a Western Blot analysis of Pichia pastoris supernatants to assess dimerization in DNASE1L3 variants. Variants with the unpaired C48 demonstrated dimerization (signal at approx. 60 kDa), but variants with mutated C48 did not show dimerization.

### DETAILED DESCRIPTION

In various aspects, the present disclosure provides recombinant proteins (e.g., recombinant proteins for human or animal therapy, including but not limited to DNase enzymes such as DIL3), which are produced by secretion from expression systems with complete processing of an N-terminal signal sequence. In various aspects, the disclosure provides methods for making such proteins and for their use in therapy.

In various aspects and embodiments, the present disclosure is based, in part, on the discovery that addition of a linker of four or more amino acids (e.g., at least 5 amino acids) between a secretion signal and a protein of interest can improve its processing and expression titers from microbial expression systems, as well as control post-translational modification profile. The present disclosure is also based, in part, on the discovery that modification of the C-terminus can improve product homogeneity profile of a protein produced in an expression system.

In some aspects, the present disclosure provides a variant of DNASE1-LIKE 3 (D1L3 variant) comprising an N-terminal extension. In some embodiments, the N-terminal extension comprises at least four amino acids. As used herein, the term “N-terminal extension” refers to an amino acid sequence that is not a secretion signal, and therefore is not removed/processed upon secretion from the host. In some embodiments, the N-terminal extension is not subject to post-translational modification by the host and/or is non-immunogenic upon administration to a human or animal subject. In exemplary embodiments, the N-terminal extension is predominately Gly residues (i.e., more than 50% Gly residues), and may have one or more Ser or Cys residues.

In some embodiments, the DIL3 variant produced by the host cell comprises a DIL3 enzyme lacking a signal peptide and comprises an amino acid sequence that has at least 80%, or at least 85%, or at least 90%, or at least 95%, or at least 97% %, or at least 98%, or at least 99% sequence identity to amino acids 21 to 282 of SEQ ID NO: 4 (Isoform 1) or amino acids 21 to 252 of SEQ ID NO: 5 (Isoform 2). When referring to sequence identity with SEQ ID NO: 4 or 5, and unless stated otherwise, sequences refer to mature enzymes lacking the signal peptide. Further, unless stated otherwise, amino acid positions are numbered with respect to the natural N-terminus of the enzyme, without signal peptide. Accordingly, for example, reference to sequence identity to the enzyme of SEQ ID NO:4 (human D1L3, Isoform 1) refers to a percent identity with the mature enzyme having M21 at the N-terminus.

In some embodiments, the D1L3 variant is produced in a host cell (such as but not limited to Pichia pastoris) by cleavage of an N-terminal signal peptide. Any signal peptide that allows for secretion of the D1L3 in the desired expression system may be used. In some embodiments, the signal peptide is a signal peptide of a natural secretary protein. In some embodiments, the signal peptide is a chimeric or synthetic signal peptide that enables protein secretion. In some embodiments, the signal peptide is a prokaryotic signal peptide. In some embodiments, the signal peptide is a microbial signal peptide. In some embodiments, the signal peptide is a eukaryotic signal peptide. In some embodiments, the signal peptide is a yeast signal peptide. In some embodiments, the signal peptide is a mammalian signal peptide. Signal peptides suitable for secretion are disclosed in U.S. Pat. Nos. 5,580,758; 6,107,057; 7,741,075; 10,435,694; 11,306,127; 11,370,815; US Patent Application Publication Nos. 2007/0117186, 2010/0055125, 2016/0168198, the disclosure of each of which is hereby incorporated by reference.

In some embodiments, the signal peptide is selected from E. coli OmpA signal peptide (SEQ ID NO: 56), E. coli DsbA signal peptide (SEQ ID NO: 67), E. coli ST-II signal peptide (SEQ ID NO: 68), E. coli FimD signal peptide (SEQ ID NO: 55), Salmonella enterica DsbA signal peptide (SEQ ID NO: 51), a synthetic Bordetella pertussis signal peptide (SEQ ID NO: 57), and synthetic signal peptide sequences (e.g., SEQ ID NOs: 49, 50, 52, 53 and 54).

In some embodiments, the signal peptide is selected from DNASE1L3 signal peptide (SEQ ID NO: 37), Alpha Mating Factor (SEQ ID NO: 38), Alpha Mating Factor Pre-Sequence (SEQ ID NO: 39), Human Serum Albumin signal peptide (SEQ ID NO: 40), Bovine DNASE1 signal peptide (SEQ ID NO: 41), Bovine DNASE1 signal peptide+Kex2-Site (SEQ ID NO: 42), Alpha Amylase signal peptide (SEQ ID NO: 43), Glucoamylase Signal Peptide (SEQ ID NO: 44), Inulinase signal peptide (SEQ ID NO: 45), Invertase signal peptide (SEQ ID NO: 46), Killer protein signal peptide (SEQ ID NO: 47), and Lysozyme signal peptide (SEQ ID NO: 48).

In some embodiments, the signal peptide is the alpha-mating factor (αMF) pre-pro secretion leader from Saccharomyces cerevisiae (SEQ ID NO: 38).

In some embodiments, the N-terminal extension is at least four, or at least five, or at least six, or at least seven, or at least eight, or at least nine, or at least ten, or at least 11, or at least 12, or at least 13, or at least 14, or at least 15 amino acids in length. In various embodiments, the N-terminal extension has a length in the range of 4 to about 18 amino acids, or from 4 to about 12 amino acids, or from 4 to about 9 amino acids, or from 4 to about 7 amino acids. For example, the N-terminal extension may be 4,5,6, 7, 8, or 9 amino acids in length. In some embodiments, the N-terminal extension is a flexible or rigid sequence. In some embodiments, the first N-terminal amino acid is not a Met residue. In some embodiments, the first amino acid residue of the N-terminal extension is not a Gly residue, which as disclosed herein can be subject to post-translational modification. In some embodiments, the last amino acid residue of the N-terminal extension is not a Ser residue, which as disclosed herein can increase immunogenicity risk. In some embodiments, the last amino acid residue of the N-terminal extension is not a polar or charged amino acid residue, such as an amino acid residue selected from Ser, Thr, Gln, Asn, Glu, Asp, Arg, His and Lys. In some embodiments, the last amino acid residue of the N-terminal extension is an amino acid selected from Gly, Ala and Val. In some embodiments, the last amino acid residue of the N-terminal extension is a Gly residue. In some embodiments, the first amino acid residue of the N-terminal extension not a Gly residue, and the last amino acid residue of the N-terminal extension is a Gly residue. In some embodiments, the N-terminal extension is predominately Gly residues (i.e., more than 50% Gly residues), consists essentially of Ser and Gly residues, or consists of Ser and Gly residues. In some embodiments, the linker comprises at least one Cysteine residue. For example, Cysteine can be placed at the N-terminus to provide for site-specific chemical conjugation, such as to polyethylene glycol, or disulfide dimerization. In some embodiments, the N-terminal extension comprises or consists of the amino acid sequence of SGGGG (SEQ ID NO: 60). In some embodiments, the N-terminal extension comprises or consists of the amino acid sequence of CGGGG (SEQ ID NO: 74). In some embodiments, the N-terminal extension comprises or consists of the amino acid sequence SGGSGGSGG (SEQ ID NO: 61). In some embodiments, the N-terminal extension comprises or consists of the amino acid sequence of SGGSGGSGGSGGSGGSGG (SEQ ID NO: 62). In some embodiments, the N-terminal extension comprises a protease cleavage site. In some embodiments, the N-terminal extension is cleavable by a coagulation pathway protease. In some embodiments, the protease is thrombin, or Factor XII or a neutrophil protease. In some embodiments, the protease is thrombin. In some embodiments, the protease cleavage site comprises the amino acid sequence LVPRG (SEQ ID NO: 64), such as the N-terminal extension represented by the sequence SGGGGLVPRGSGGGG (SEQ ID NO: 65).

In some embodiments, the N-terminal extension does not include a consensus sequence for myristoylation. In some embodiments, the N-terminal extension does not comprise a consensus sequence for one or more of protein acetylation, propionylation, methylation, myristoylation, palmitoylation, ubiquitylation, and a protease cleavage site, to avoid unwanted post-translational modifications. In alternative embodiments, the N-terminal extension comprises a consensus sequence for one or more of protein acetylation, propionylation, methylation, myristoylation, palmitoylation, ubiquitylation, and a protease cleavage site, where these modifications are desired. In some embodiments, the N-terminal extension comprises an amino acid having a chemical group suitable for a chemical conjugation, the chemical group being selected from a thiol group, amino group, amido group, and carboxyl group.

In some embodiments, the N-terminal extension is non-immunogenic. In some embodiments, the junction of the N-terminal extension and sequence originating from the desired protein (such as but not limited to DIL3) is non-immunogenic as indicated by an in silico immunogenicity prediction algorithm (without limitation, e.g., the Epibase® In Silico and In Vitro Immunogenicity Platform of Lonza Group AG). In some embodiments, the last amino acid residue of the N-terminal extension is not a Ser residue. In some embodiments, the last amino acid residue of the N-terminal extension is not a polar or charged amino acid residue selected from Ser, Thr, Gln, Asn, Glu, Asp, Arg, His and Lys. In some embodiments, the last amino acid residue of the N-terminal extension is an amino acid selected from Gly, Ala and Val. In some embodiments, the last amino acid residue of the N-terminal extension is a Gly residue.

In some embodiments, the N-terminal extension improves expression titer of the D1L3 variant by at least 25%, or at least 50%, or at least 100%, or at least 150%, or at least 200%, or at least 250%, or at least 300%, or at least 350%, or at least 400%, or at least 500%, or more, compared to the same D1L3 sequence lacking the N-terminal extension, and with regard to a selected signal sequence and expression host. In some embodiments, the signal peptide is completely removed from the DIL3 variant upon secretion from the host.

In some embodiments, the DIL3 variant further comprises a C-terminal extension reducing heterogeneity. In some embodiments, the C-terminal extension is at least two, or at least three, or at least four, or at least five amino acids in length. In some embodiments, the extension is a hydrophilic sequence of 2, 3, or 4 amino acids. In some embodiments, the C-terminal amino acid is a lysine (Lys) or an arginine (Arg). In some embodiments, the C-terminal extension comprises or consists of the amino acid sequence SSR, which can be employed in some embodiments for DIL3 variants having whole or partial deletions of the C-terminal basic domain (as described further herein). In some embodiments, the DIL3 variant comprises the C-terminal basic domain having the amino acid sequence SSRAFTNSKKSVTLRKKTKSKRS (SEQ ID NO: 75), or a modified version thereof having from one to five amino acid modifications independently selected from amino acid substitutions, deletions, and insertions. When these DIL3 enzymes are expressed in Pichia pastoris the resulting polypeptide will be cleaved after the SSR sequence at the beginning of the basic domain sequence.

In some aspects, the present disclosure provides a variant of DNASE1-LIKE 3 (D1L3 variant) comprising a C-terminal extension as described above. In some embodiments, the C-terminal extension reduces heterogeneity observed for D1L3 variants having the basic domain deleted. In some embodiments, the heterogeneity is caused by a post-translational modification. In some aspects, the present disclosure provides a DIL3 variant comprising an N-terminal extension as already described and a C-terminal extension reducing heterogeneity. In some embodiments, the C-terminal extension comprises or consists of the amino acid sequence SSR.

In some embodiments, the C-terminal extension comprises an amino acid having a chemical group suitable for a chemical conjugation, the chemical group being selected from a thiol group, amino group, amido group, and carboxyl group. In some embodiments, the C-terminal extension does not comprise a consensus sequence for one or more of protein acetylation, propionylation, methylation, myristoylation, palmitoylation, ubiquitylation, and a protease cleavage site to avoid undesired post-translational modifications. In alternative embodiments, the C-terminal extension comprises a consensus sequence for one or more of protein acetylation, propionylation, methylation, myristoylation, palmitoylation, ubiquitylation, and a protease cleavage site, where post-translational modification is desired. In some embodiments, the C-terminal extension is non-immunogenic. In some embodiments, the junction of the C-terminal extension and sequence originating from DIL3 is non-immunogenic.

In some embodiments, the D1L3 variant comprises a deletion of at least three, or at least five, or at least eight, or at least ten, or at least twelve, or at least fifteen, or at least eighteen, or at least twenty, or at least twenty-one, or all 23 amino acids of a C-terminal basic domain (BD) defined by amino acids 283 to 305 of SEQ ID NO: 4. In some embodiments, the DIL3 variant comprises a deletion of at least three amino acids of the C-terminal basic domain (BD) defined by amino acids 283 to 305 of SEQ ID NO: 4, wherein the D1L3 variant comprises the C-terminal extension of any of the embodiments disclosed here (without limitation, e.g., having the amino acid sequence SSR). In such embodiments, the C-terminus with SSR extension is equivalent to a 20 amino acid deletion of the basic domain.

D1L3 features a 23-amino acid long C-terminal tail defined by amino acids 283 to 305 of SEQ ID NO: 4, which contains 9 basic amino acids and is thus known a basic domain (BD). The BD is unique to DIL3 and is not present in DNASE 1 (D1). The BD contains a nuclear localization signal (NLS) that is believed to target the enzyme to the nucleus during apoptosis. While it has been widely considered that the BD is also critical for the biologic activity of D1L3 in the extracellular space, deletion of the C-terminal tail in fact stimulates chromatinase activity of DIL3. In some embodiments, the D1L3 variant comprises a BD having the sequence SSRAFTNSKKSVTLRKKTKSKRS (SEQ ID NO: 75) or a derivative thereof having a truncation of at least three, or at least five, or at least eight, or at least ten, or at least twelve, or at least fifteen, or at least eighteen amino acids, or about 20 amino acids.

In some embodiments, the D1L3 variant comprises a mutation (e.g., a substitution, insertion or a deletion) of three clusters of paired basic amino acids of unknown function, i.e. K291/K292, R297/K298/K299, and K303/R304 corresponding to SEQ ID NO: 4. In some embodiments, the DIL3 variant comprises a BD having the sequence SSRAFTNSKKSVTLRKKTKSKRS (SEQ ID NO: 75) or a derivative thereof having from one to five amino acid modifications altering the paired basic amino acids. In some embodiments, the D1L3 variant comprises a truncation of the BD the deletes at least one of the paired basic amino acids. In some embodiments, the DIL3 variant exhibits a reduced proteolytic cleavage at these paired basic amino acids. In some embodiments, the DIL3 variant comprises the N-terminal extension of any of the embodiments disclosed herein (without limitation, e.g., comprising the amino acid sequence SGGGG (SEQ ID NO: 60) or CGGGG (SEQ ID NO: 74)) and/or the D1L3 variant comprises the C-terminal extension of any of the embodiments disclosed here (without limitation, e.g., having the amino acid sequence SSR). In some embodiments, the C-terminal extension is added in place of the C-terminal basic domain.

In various embodiments, the D1L3 variant comprises an amino acid sequence that has at least 70% sequence identity to D1L3 Isoform 1 (SEQ ID NO: 4) or DIL3 Isoform 2 (SEQ ID NO: 5) lacking the BD (i.e., at least 80% sequence identity with amino acids 21 to 282 of SEQ ID NO: 4, or amino acids 21 to 252 of SEQ ID NO: 5), and wherein the DIL3 variant has a deletion of one or more amino acids from the BD. Amino acid deletions of the Basic Domain of D1L3 improve its chromatin-degrading activity. Further, increasing deletions of the 23-amino acid BD directly correlate with increasing chromatin-degrading activity. In some embodiments, the DIL3 variant having a deletion of at least one amino acid, or at least 3, or at least 5, or at least 8, or at least 9, or at least 13, or at least 14 or at least 15, or at least 18, or about 20 C-terminal amino acids of the DIL3 basic domain have increased ability to degrade mono-nucleosomes. In some embodiments, the DIL3 variant comprises the N-terminal extension of any of the embodiments disclosed herein (without limitation, e.g., comprising or consisting of the amino acid sequence SGGGG (SEQ ID NO: 60) or CGGGG (SEQ ID NO: 74)) and/or the D1L3 variant comprises the C-terminal extension of any of the embodiments disclosed here (without limitation, e.g., having the amino acid sequence SSR).

In various embodiments, the amino acid deletions from the BD are at the C-terminus of the BD. For example, the DIL3 variant may have a deletion of at least the five C-terminal amino acids of the BD. In some embodiments, the DIL3 variant has a deletion of at least the eight C-terminal amino acids of the BD. In some embodiments, the D1L3 variant has a truncation of at least the eight C-terminal amino acids of the BD. In some embodiments, the DIL3 variant has a deletion of at least the ten C-terminal amino acids of the BD. In some embodiments, the DIL3 variant has a truncation of at least the ten C-terminal amino acids of the BD. In some embodiments, the DIL3 variant has a deletion of at least the twelve C-terminal amino acids of the BD. In some embodiments, the DIL3 variant has a truncation of at least the twelve C-terminal amino acids of the BD. In some embodiments, the DIL3 variant has a deletion of at least the fifteen C-terminal amino acids of the BD. In some embodiments, the D1L3 variant has a truncation of at least the fifteen C-terminal amino acids of the BD. In some embodiments, the DIL3 variant has a deletion of at least the eighteen C-terminal amino acids of the BD. In some embodiments, the DIL3 variant has a truncation of at least the eighteen C-terminal amino acids of the BD. In some embodiments, the DIL3 variant has a deletion of at least the twenty C-terminal amino acids of the BD. In some embodiments, the D1L3 variant has a truncation of at least (or about) the twenty C-terminal amino acids of the BD. In some embodiments, the D1L3 variant has a deletion of at least the twenty-three C-terminal amino acids of the BD. In some embodiments, the D1L3 variant has a truncation of at least the twenty-three C-terminal amino acids of the BD. In some embodiments, the DIL3 variant has a deletion of at least the C-terminal serine of the BD. In some embodiments, the DIL3 variant has a truncation of C-terminal serine of the BD. In some embodiments, the DIL3 variant comprises the N-terminal extension of any of the embodiments disclosed herein (without limitation, e.g., comprising the amino acid sequence SGGGG (SEQ ID NO: 60) or CGGGG (SEQ ID NO: 74)) and/or the D1L3 variant comprises the C-terminal extension of any of the embodiments disclosed here (without limitation, e.g., having the amino acid sequence SSR).

Alternatively, deletions of the BD (e.g., from three to 23 amino acids) can be anywhere in the BD, and not necessarily from the C-terminus of the BD. For example, in various embodiments, the DIL3 variant has 1, 2, 3, 4, 5, 6, 7, 8, 9, 10, 11, 12, 13, 14, 15, 16, 17, 18, 19, 20, 21, 22, or 23 amino acids deleted from the BD. In some embodiments, the D1L3 variant has a deletion of at least 1, or at least 3, or at least 5, or at least 8, or at least 12, or at least 15, or at least 18, or at least 21 amino acids from the BD. In some embodiments, the DIL3 variant has a truncation of at least 1, or at least 3, or at least 5, or at least 8, or at least 12, or at least 15, or at least 18, or at least 20 amino acids, or at least 21 amino acids from the BD. These deletions can be independently selected from the N-terminal side of the BD, from the C-terminal side of the BD, and internal to the BD. In some embodiments, one or more amino acid deletions are within the NLS. In some embodiments, the deleted amino acid is the C-terminal serine of the BD. In some embodiments, the deletion is sufficient to remove all paired basic amino acids in the BD from the enzyme. In some embodiments, the DIL3 variant comprises the N-terminal extension of any of the embodiments disclosed herein (without limitation, e.g., comprising the amino acid sequence SGGGG (SEQ ID NO: 60) or CGGGG (SEQ ID NO: 74)) and/or the D1L3 variant comprises the C-terminal extension of any of the embodiments disclosed here (without limitation, e.g., having the amino acid sequence SSR). In some embodiments, a C-terminal extension of the sequence SSR is equivalent to a truncation of 20 amino acids of the BD.

In addition to deletions of one or more amino acids, the BD may further comprise amino acid substitutions, which may further impact chromatin-degrading activity. For example, the DIL3 variant may have from 1 to 20 amino acid substitutions of BD amino acids, in addition to a deletion of at least three amino acids. In some embodiments, the BD contains a substitution of at least three amino acids, or at least five amino acids, or at least 10 amino acids. In some embodiments, at least two amino acid substitutions are in the NLS of the BD. In some embodiments, one or more paired basic amino acids in the BD are substituted to prevent cleavage. In such embodiments, a more homogeneous enzyme may be expressed and secreted, e.g., for recombinant enzyme production. In some embodiments, the D1L3 variant comprises the N-terminal extension of any of the embodiments disclosed herein (without limitation, e.g., comprising the amino acid sequence SGGGG (SEQ ID NO: 60) or CGGGG (SEQ ID NO: 74)) and/or the D1L3 variant comprises the C-terminal extension of any of the embodiments disclosed here (without limitation, e.g., having the amino acid sequence SSR).

In some embodiments, the D1L3 variant has a deletion of one or more additional amino acids from the C-terminus, in addition to a deletion of the BD. For example, the DIL3 variant may have a deletion of an additional one to fifty amino acids, or from one to twenty amino acids, or from one to ten amino acids, or from one to five amino acids from the C-terminal amino acids of SEQ ID NO: 4 or SEQ ID NO: 5, in addition to the deletion of the BD. In some embodiments, the D1L3 variant comprises the N-terminal extension of any of the embodiments disclosed herein (without limitation, e.g., comprising the amino acid sequence SGGGG (SEQ ID NO: 60) or CGGGG (SEQ ID NO: 74)) and/or the DIL3 variant comprises the C-terminal extension of any of the embodiments disclosed here (without limitation, e.g., having the amino acid sequence SSR).

In some embodiments, after partial or complete deletion of the BD as described, from 1 to 10 amino acids, or from 1 to 5 amino acids may be added to the C-terminus that do not impact chromatin-degrading activity. In some embodiments, the addition of the N-terminal extension of any of the embodiments disclosed herein (without limitation, e.g., comprising the amino acid sequence SGGGG (SEQ ID NO: 60) or CGGGG (SEQ ID NO: 74)) and/or the C-terminal extension of any of the embodiments disclosed here (without limitation, e.g., having the amino acid sequence SSR) do not impact chromatin-degrading activity.

In some embodiments, the DIL3 variant comprises a substitution of C68 and/or C194 with respect to SEQ ID NO: 4 (C48 and C174 when numbered without the signal peptide). In some embodiments, the mutation is selected from C68S, C68A, C68G, C194S, C194A, and C194G with respect to SEQ ID NO: 4. In some embodiments, the DIL3 variant comprises one or more mutations that result in resistance to proteolysis by one or more of plasmin, thrombin, trypsin, and proteases produced by mammalian and non-mammalian cell lines. In some embodiments, the D1L3 variant has one or more mutations of amino acid residues selected from K180, K200, K259, and R285 with respect to SEQ ID NO: 4. In some embodiments, the DIL3 variant has one or more mutations of amino acid residues selected from R22, R29, K45, K47, K74, R81, R92, K107, K176, R212, R226, R227, K250, K259, and K262 with respect to SEQ ID NO: 4.

In some embodiments, the D1L3 variant comprises one or more unpaired cysteines which are configured for and/or capable of dimerization, for example, an unpaired cysteine at position 48 relative to the amino acid sequence of SEQ ID NO: 4 (numbered without the signal peptide). In some embodiments, the disclosure provides a D1L3 dimer according to this disclosure that is dimerized through disulfide bridge at C48. In some embodiments, the D1L3 variant comprises the incorporation of non-native cysteines, which can contribute to dimerization, including incorporation of non-native cysteines at the N-terminus. In such embodiments, the disclosure provides a D1L3 dimer, which can be used in therapy according to the disclosure. In some embodiments, the DIL3 variant comprises a substitution of cysteine at position 48 (C48) with respect to SEQ ID NO: 4, to further control dimerization positions. For example, the mutation can be selected from C48A, C48G, and C48S. In some embodiments, the substitution of C48 (e.g., to C48A or C48G) increases the enzymatic capacity (such as chromatin degradation) of the DIL3 enzyme. In some embodiments, the D1L3 variant has a C-terminal extension which comprises or consists of the amino acid sequence SSR and a mutation of C48A with respect to SEQ ID NO: 4 (numbered without the signal peptide).

In some embodiments, the D1L3 variant comprises: (i) a D1L3 enzyme lacking a signal peptide and comprising an amino acid sequence that has at least 80%, or at least 85%, or at least 90%, or at least 95%, or at least 97% %, or at least 98%, or at least 99% sequence identity to amino acids 21 to 282 of SEQ ID NO: 4 or amino acids 21 to 252 of SEQ ID NO: 5; (ii) a deletion of at least three amino acids of the C-terminal basic domain (BD) defined by amino acids 283 to 305 of SEQ ID NO: 4; and (iii) an N-terminal extension of at least four amino acids as described herein. In some embodiments, the DIL3 variant further comprises a substitution of C48 with respect to SEQ ID NO: 4. In some embodiments, the mutation is selected from C48S or C48A with respect to SEQ ID NO: 4 (numbered without the signal peptide). In some embodiments, the DIL3 variant further comprises a modification (without limitation, e.g., Pegylation) of C48 and/or C174 with respect to SEQ ID NO: 4.

In some embodiments, the D1L3 variant comprises: (i) a D1L3 enzyme lacking a signal peptide and comprising an amino acid sequence that has at least 80%, or at least 85%, or at least 90%, or at least 95%, or at least 97% %, or at least 98%, or at least 99% sequence identity to amino acids 21 to 282 of SEQ ID NO: 4 or amino acids 21 to 252 of SEQ ID NO: 5; (ii) a deletion of at least three amino acids of the C-terminal basic domain (BD) defined by amino acids 283 to 305 of SEQ ID NO: 4; and (iii) a C-terminal extension comprising the amino acid sequence SSR. In some embodiments, the DIL3 variant further comprises a substitution of C48 and/or C174 with respect to SEQ ID NO: 4 (numbered without the signal peptide). In some embodiments, the mutation is selected from C48S or C48A with respect to SEQ ID NO: 4. In some embodiments, the D1L3 variant further comprises a modification (without limitation, e.g., Pegylation) of C48 and/or C174 with respect to SEQ ID NO: 4.

In some embodiments, the DIL3 variant comprises: (i) a mature D1L3 enzyme lacking a signal peptide and comprising an amino acid sequence that has at least 80%, or at least 85%, or at least 90%, or at least 95%, or at least 97% %, or at least 98%, or at least 99% sequence identity to amino acids 21 to 282 of SEQ ID NO: 4 or amino acids 21 to 252 of SEQ ID NO: 5; (ii) a deletion of at least three amino acids of the C-terminal basic domain (BD) defined by amino acids 283 to 305 of SEQ ID NO: 4; (iii) the N-terminal extension described herein; and (iv) a C-terminal extension comprising the amino acid sequence SSR. In some embodiments, the DIL3 variant further comprises a substitution of C48 and/or C174 with respect to SEQ ID NO: 4 (numbered without the signal peptide). In some embodiments, the mutation is selected from C48S and C48A with respect to SEQ ID NO: 4. In some embodiments, the DIL3 variant further comprises a modification (without limitation, e.g., PEGgylation) of C48 and/or C174 with respect to SEQ ID NO: 4.

In some embodiments, the DIL3 variant has one or more mutations of serine residues, such as those selected from S91C, S131C, and S253C with respect to SEQ ID NO: 4 (numbering includes the signal peptide).

Exemplary D1L3 variants in accordance with this disclosure include SEQ ID NO: 63, SEQ ID NO: 66, SEQ ID NO: 70, SEQ ID NO: 71, SEQ ID NO: 72, SEQ ID NO: 73, or SEQ ID NO: 77, where the signal peptide is not present and is completely processed by the host expression system. SEQ ID NO: 63 employs the N-terminal extension of SEQ ID NO: 60 to accomplish signal peptide processing, and contains a full basic domain deletion. SEQ ID NO: 66 further includes a C48S substitution, and an SSR C-terminal extension (as compared to SEQ ID NO: 63). SEQ ID NO: 70 exemplifies a C48A substitution (but otherwise has the sequence of SEQ ID NO: 66). SEQ ID NO: 71 is similar to SEQ ID NO: 70, but with a CGGGG (SEQ ID NO: 74) linker before the signal peptide. SEQ ID NO: 72 exemplifies the C48S substitution with the CGGGG (SEQ ID NO: 74) linker. SEQ ID NO: 73 exemplifies the C48A substitution with CGGGG (SEQ ID NO: 74) linker and full basic domain. When produced in Pichia pastoris the polypeptide of SEQ ID NO: 73 will have SSR at the C-terminus (e.g., 20 amino acids of the basic domain will be cleaved). SEQ ID NO: 77 employs a C48A substitution, a SGGGG (SEQ ID NO: 60) linker, and a C-terminal extension (SSR) added after deletion of an additional 12 amino acids (in addition to the basic domain deletion).

In some embodiments, the D1L3 variant comprises a fusion or conjugation to a half-life extending moiety. In some embodiments, the half-life extending moiety is a polymer. In some embodiments, the polymer is a polyethylene glycol (PEG). In some embodiments, the PEG is conjugated to the N-terminus (optionally within the N-terminal extension of any of the embodiments disclosed herein, e.g., comprising the amino acid sequence SGGGG (SEQ ID NO: 60) or CGGGG (SEQ ID NO: 74)). In some embodiments, the PEG polymer connects two D1L3 variant molecules through their N-termini. In some embodiments, the PEG is conjugated to the C-terminus (optionally within the C-terminal extension of any of the embodiments disclosed here, e.g., having the amino acid sequence SSR). In some embodiments, the DIL3 comprises the basic domain, and PEG is conjugated to the basic domain. In some embodiments, the PEG polymer connects two D1L3 variant molecules through their C-termini.

In some embodiments, the PEG polymer is conjugated to one or more amino acids within positions corresponding to R95 to V126 of SEQ ID NO: 4. In some embodiments, one or more PEGylated amino acids are selected from lysine, cysteine, histidine, arginine, aspartic acid, glutamic acid, serine, threonine and tyrosine, optionally wherein one or more PEGylated amino acids are introduced by substitution of one or more amino acids between R95 and V126 relative to SEQ ID NO: 4.

In some embodiments, the one or more amino acids are PEGylated by: (a) PEGylation of lysine (Lys or K) conducted via amine conjugation; (b) PEGylation of glutamine conducted via transglutaminase (TGase) mediated enzymatic conjugation; and/or (c) PEGylation of cysteine (Cys or C) conducted via thiol conjugation. In some embodiments, one or more PEGylated amino acids are conjugated with PEG moieties that are independently selected from a linear or branched PEG having molecular weights that are independently selected and in the range of about 2 kDa to about 60 kDa, or about 5 kDa to about 30 kDa. PEGylation is disclosed in WO 2019/036719 and WO 2020/076817, both of which are hereby incorporated by reference in its entirety.

In these embodiments, the PEG moiety will provide a half-life extension property, while avoiding disulfide scrambling and/or protein misfolding. In some embodiments, the PEG moiety is conjugated through maleimide chemistry, which can be conducted under mild conditions. Other conjugation chemistries are known and may be used, such as vinyl sulfone, dithyopyridine, and iodoacetamide activation chemistries.

In some embodiments, the DIL3 variant comprises a fusion or conjugation to a half-life extending moiety. In some embodiments, the half-life extending moiety is a fusion partner. In some embodiments, the fusion partner is selected from albumin, transferrin, an Fc, or elastin-like protein, XTEN sequence, or a variant thereof. In some embodiments, the fusion partner is an albumin. In some embodiments, the fusion partner is a human albumin comprising an amino acid sequence that is at least 80% identical to SEQ ID NO: 26. In some embodiments, the human albumin comprises at least one of E505Q, T527M, K573P substitutions with respect to SEQ ID NO: 26. In some embodiments, the human albumin comprises at least two of E505Q, T527M, K573P substitutions with respect to SEQ ID NO: 26. In some embodiments, the human albumin comprises each of E505Q, T527M, K573P substitutions with respect to SEQ ID NO: 26. In some embodiments, the fusion partner is fused at the N-terminus to mature D1L3 enzyme lacking a signal peptide (and through a linker sequence) and comprising an amino acid sequence that has at least 80%, or at least 85%, or at least 90%, or at least 95%, or at least 97% %, or at least 98%, or at least 99% sequence identity to amino acids 21 to 282 of SEQ ID NO: 4 or amino acids 21 to 252 of SEQ ID NO: 5 and having a deletion of at least three amino acids of the C-terminal basic domain (BD) defined by amino acids 283 to 305 of SEQ ID NO: 4. In some embodiments, the D1L3 variant comprises the amino acid sequence of SEQ ID NO: 76 or SEQ ID NO: 78, or a variant thereof having at least about 99% sequence identity thereto. In some embodiments, the N-terminal extension of any of the embodiments disclosed herein links the fusion partner with the N-terminus of mature D1L3 enzyme. In some embodiments, the fusion partner is fused to the mature D1L3 enzyme via a linker adjoining the fusion partner and the mature D1L3 enzyme. In some embodiments, the N-terminal extension of any of the embodiments disclosed herein links the fusion partner with the N-terminus of mature D1L3 enzyme. In some embodiments, the linker is a flexible or rigid linker, and/or comprises a protease cleavage site. In some embodiments, the linker is cleavable by a coagulation pathway protease. In some embodiments, the protease is Factor XII or a neutrophil protease. In some embodiments, the protease is thrombin. In some embodiments, the protease cleavage site comprises the amino acid sequence LVPRG (SEQ ID NO: 64), such as the linker represented by the sequence SGGGGLVPRGSGGGG (SEQ ID NO: 65). In some embodiments, the linker is from about 5 to about 50 amino acids, or from about 10 to about 35 amino acids, or from about 15 to about 35 amino acids in length. In some embodiments, the linker comprises the amino acid sequence S(GGS)₄GSS (SEQ ID NO: 23), S(GGS)₉GSS (SEQ ID NO: 24), and (GGS)₉GS (SEQ ID NO: 25). In some embodiments, the linker comprises the sequence (GGGGS)₅GGGG (SEQ ID NO: 79), as shown by the fusion protein of SEQ ID NO: 76.

In some embodiments, the DIL3 variant comprises a flexible linker between the D1L3 sequence and the half-life extending moiety. Flexible linkers are predominately or entirely composed of small, non-polar or polar residues such as Gly, Ser and Thr. An exemplary flexible linker comprises (Gly_{y}Ser)ₙS_{z} linkers, where y is from 1 to 10 (e.g., from 1 to 5), n is from 1 to about 10, and z is 0 or 1. In some embodiments, n is from 3 to about 8, or from 3 to about 6. In exemplary embodiments, y is from 2 to 4, and n is from 3 to 8. Due to their flexibility, these linkers are unstructured. More rigid linkers include polyproline or poly Pro-Ala motifs and α-helical linkers. An exemplary α-helical linker is A(EAAAK)ₙA, where n is as defined above (e.g., from 1 to 10, or 3 to 6). Generally, linkers can be predominately composed of amino acids selected from Gly, Ser, Thr, Ala, and Pro. Exemplary linker sequences contain at least 10 amino acids, and may be in the range of 10 to about 50 amino acids, or about 15 to about 40 amino acids, or about 15 to about 35 amino acids. Exemplary linker designs are provided as SEQ ID NOS: 18 to 25.

In some embodiments, the DIL3 variant with fusion partner comprises a linker, wherein the amino acid sequence of the linker is predominately glycine and serine residues, or consists essentially of, or consists of glycine and serine residues. In some embodiments, the ratio of Ser and Gly in the linker is, respectively, from about 1:1 to about 1:10, from about 1:2 to about 1:6, or about 1:4. Exemplary linker sequences comprise or consist of S(GGS)₄GSS (SEQ ID NO: 23), S(GGS)₉GSS (SEQ ID NO: 24), and (GGS)₉GS (SEQ ID NO: 25). In some embodiments, the linker has at least 10 amino acids, or at least 15 amino acids, or at least 20 amino acids, or at least 25 amino acids, or at least 30 amino acids. For example, the linker may have a length of from 15 to 40 amino acids. In various embodiments, longer linkers of at least 15 amino acids can provide improvements in titer upon expression in Pichia pastoris.

In some aspects, the present disclosure provides a method for expressing the DNase1-like 3 variant of any one of the embodiments disclosed herein. In some embodiments, the method comprises introducing a genetic construct encoding the DNase1-like 3 variant of any of the embodiments disclosed herein and comprising a signal peptide in a yeast cell, and recovering the DNase1-like 3 variant. In some embodiments, the yeast cell is Pichia pastoris. In some embodiments, the signal peptide is the alpha-mating factor (αMF) pre-pro secretion leader from Saccharomyces cerevisiae (SEQ ID NO: 38). In some embodiments, the fusion protein is synthesized with an N-terminal signal peptide. The signal peptide may be completely removed during secretion from the host cell. With respect to expression in Pichia pastoris, the alpha-mating factor (αMF) pre-pro secretion leader from Saccharomyces cerevisiae (SEQ ID NO: 38) may be used for expression. These elements are cleaved during expression, and are not present in the DIL3 variant enzyme product.

In some aspects, the present disclosure provides an isolated polynucleotide encoding the D1L3 variant of any of the embodiments disclosed herein, and which provide advantages for expression ex vivo or in vivo. In some aspects, the present disclosure provides a polynucleotide that is an mRNA or a modified mRNA (mmRNA). In some aspects, the present disclosure provides a polynucleotide that is DNA. The disclosure in some aspects provides pharmaceutical compositions comprising the DIL3 enzyme described herein, or optionally a polynucleotide encoding the DIL3 enzyme, or a transfection or expression vector comprising the same, or a cell comprising the polynucleotide or vector, and a pharmaceutically acceptable carrier.

In some embodiments, delivery of polynucleotides is used for therapy. Encoding polynucleotides can be delivered as mRNA or as DNA constructs using known procedures, e.g., electroporation or cell squeezing, and/or vectors (including viral vectors). mRNA polynucleotides can include known modifications (mmRNA) to avoid activation of the innate immune system. See WO 2014/028429, which is hereby incorporated by reference in its entirety. In some embodiments, the polynucleotide is delivered to the body of a subject. In some embodiments, the polynucleotides is delivered into a cell in vitro, and the cell is delivered to the body of a subject. The cell can be, for example, a white blood cell (e.g., a T cell, B cell, or macrophage), an endothelial cell, an epithelial cell, a hepatocyte, a fibroblast, or a stem cell (e.g., a hematopoietic stem cell).

In some embodiments, the polynucleotide used for therapy is a modified mRNA (mmRNA). In some embodiments, the mmRNA is administered to a subject in need of treatment. In some embodiments, the cells are transformed with a modified mRNA (mmRNA) in vitro or ex vivo, expanded before or after transfection, and used for therapy (cell therapy). In some embodiments, the mmRNAs may be uniformly modified along the entire length of the molecule. In alternative embodiments, the mmRNAs may not be uniformly modified along the entire length of the molecule. Different nucleotide modifications and/or backbone structures may exist at various positions in the nucleic acid.

In some embodiments, the nucleotide analogs or other modification(s) may be located at any position(s) of a nucleic acid such that the function of the nucleic acid is not substantially decreased. In some embodiments, the mmRNAs may comprise a 5′ or 3′ terminal modification.

In some embodiments, the mmRNA may contain at least about 5% modified nucleotides, or at least about 10% modified nucleotides, or at least about 20% modified nucleotides, or at least about 50% modified nucleotides, at least about 80% modified nucleotides. In some embodiments, the mmRNA may contain less than about 10% modified nucleotides, or less than about 20% modified nucleotides, or less than about 50% modified nucleotides.

In some embodiments, the mmRNA may include a polynucleotide modification such as, but not limited to, a nucleoside modification. The nucleoside modification may include, but is not limited to, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2, N2-dimethyl-6-thio-guanosine, and combinations thereof. Suitable modifications are disclosed in US20190060458, the contents of which are hereby incorporated by reference in its entirety.

In some aspects, the present disclosure provides a vector for introducing the polynucleotide of any of embodiments disclosed herein to a host cell. In some aspects, the present disclosure provides a host cell comprising the vector of any of embodiments disclosed herein.

In some embodiments, the polynucleotide used for therapy is a DNA molecule encoding a wild type D1L3 enzyme or any variant of DIL3 disclosed herein (i.e., gene therapy). In some embodiments, the cells are transformed with a DNA molecule encoding a wild type D1L3 enzyme or any variant of DIL3 disclosed herein in vitro or ex vivo, expanded, and used for therapy (i.e., cell therapy). In some embodiments, the DNA molecule is a vector. A vector generally comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In some embodiments, the vector is a viral vector. Exemplary vectors include autonomously replicating plasmids or a virus (e.g. AAV vectors). The term should also be construed to include non-plasmid and non-viral compounds that facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

In some embodiments, the polynucleotide or cell therapy may employ expression vectors, which comprise the nucleic acid encoding a chromatinase (e.g., DIL3) operably linked to an expression control region that is functional in the host cell. The expression control region is capable of driving expression of the operably linked encoding nucleic acid such that the chromatinase is produced in a human cell transformed with the expression vector. Expression control regions are regulatory polynucleotides (sometimes referred to herein as elements), such as promoters and enhancers, that influence expression of an operably linked nucleic acid. An expression control region of an expression vector is capable of expressing operably linked encoding nucleic acid in a human cell. In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. In various embodiments, the chromatinase expression is inducible or repressible. Typically, the amount of increase or decrease conferred by such elements is proportional to the amount of signal present; the greater the amount of signal, the greater the increase or decrease in expression.

In some embodiments, the viral vector is an adeno-associated viral vector (AAV). In some embodiments, suitable AAV-based vectors in the current disclosure have very limited capacity to induce immune responses in humans. The AAV genome is typically built of single-stranded deoxyribonucleic acid (ssDNA), either positive- or negative-sensed, which is about 4.7 kilobases long. The AAV genome comprises inverted terminal repeats (ITRs) at both ends of the DNA strand, and two open reading frames (ORFs): rep and cap. Development of AAVs as gene therapy vectors has eliminated the integrative capacity of the vector by removal of the rep and cap from the DNA of the vector. In some embodiments, a gene encoding a wild type D1L3 enzyme or any variant of DIL3 disclosed herein, which is operably linked to a promoter, may be inserted between the inverted terminal repeats (ITR). In some embodiments, the AAV vector comprising a wild type D1L3 enzyme or any variant D1L3 disclosed herein may form a concatemer in the nucleus after the single-stranded vector DNA is converted by host cell DNA polymerase complexes into double-stranded DNA. In some embodiments, the AAV vector comprising a wild type DIL3 enzyme or any variant D1L3 disclosed herein may thus form episomal concatemers in the host cell nucleus. In some embodiments, the concatemers may remain intact for the life of the non-dividing host cell. In some embodiments, the concatemers may be lost through cell division dividing cells.

In an illustrative embodiment, the AAV serotype 8 (AAV2/8) vector is used. In some embodiments, the recombinant AAV serotypes used for delivery of the polynucleotide are replication-defective, generally do not insert into the host genome and show a lack of pathogenicity and immune response in human subjects. Any AAV vector may be used, including, but not limited to, AAV1, AAV2, AAV3, AAV4, AAV5, AAV6, AAV7, AAV8 and combinations thereof. In some instances, the AAV comprises LTRs that are of a heterologous serotype in comparison with the capsid serotype (e.g., AAV2 ITRs with AAV5, AAV6, or AAV8 capsids).

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription-initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), FIp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family, and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences including CRISPR/CAS9, zinc finger, TALEN, and meganuclease gene-editing technologies.

Thus, in some embodiments, the invention provides mammalian host cells (e.g., human host cells), as well as methods of making and using the same. The host cells comprise a heterologous polynucleotide encoding a chromatinase enzyme (as described). The host cells delivered to a subject express and secrete the encoded chromatinase enzyme. In these aspects, challenges in manufacturing chromatinases such as D1L3 at large scale are avoided. Further, by expressing and delivering DIL3 through heterologous expression in a white blood cell such as a T cell, B cell or macrophage, or a fibroblast, DIL3 therapy can be localized in part to areas of inflammation or tissue destruction or cell apoptosis or wound healing. Further, since WT D1L3 has a circulation half-life of less than about 30 minutes, the cell therapy described herein provides for a sustained therapy, with as few as one, two, three, or four treatments in some embodiments. In some embodiments, the therapy is provided to a subject for treatment of cancer (e.g., leukemia) or viral infection, including infection of the lower respiratory tract. In some embodiments, the host cells are created from cells of the subject to be treated or an HLA-matched donor. In some embodiments, the cells are HLA null, or are created from HLA-matched source cells.

In some aspects, the present disclosure provides a pharmaceutical composition comprising an effective amount of the DIL3 variant of any of the embodiments disclosed herein, the D1L3 variant produced according the method of any of the embodiments disclosed herein, the polynucleotide according to any of the embodiments disclosed herein, the vector according to any of the embodiments disclosed herein, or the host cell according to any of the embodiments disclosed herein, and a pharmaceutically acceptable carrier.

In some embodiments, the composition comprises the DIL3 variant of any of the embodiments disclosed herein, and a pharmaceutically acceptable carrier for parenteral administration. In some embodiments, the pharmaceutical composition is formulated for topical, parenteral, or pulmonary administration. In some embodiments, the pharmaceutical composition is formulated for intradermal, intramuscular, intraperitoneal, intraarticular, intravenous, subcutaneous, intraarterial, ocular, oral, sublingual, pulmonary, or transdermal administration.

In embodiments, the method for recombinant production of variants of DIL3 enzyme employs a non-mammalian expression system, e.g., a eukaryotic non-mammalian expression system, such as Pichia pastoris. In embodiments, the Pichia pastoris encodes the DNase enzyme with its native signal peptide allowing for secretion from host cells. In embodiments, the expression system is a mammalian cell expression system, such as Chinese Hamster Ovary (CHO) cells. In embodiments, the method for recombinant production of variants of D1L3 enzyme further comprises isolating and/or purifying the DIL3 enzyme and subjecting the isolated and/or purified the DIL3 enzyme to a modification. In embodiments, the modification comprises conjugation of the isolated and/or purified the DIL3 enzyme to a polymer (without limitation, e.g., PEG). In embodiments, the polymer is added to a specific site using the desired conjugation chemistry (without limitation, e.g., maleimide chemistry).

In other aspects, the present disclosure provides a method for treating a subject in need of extracellular chromatin degradation, extracellular trap (ET) degradation and/or neutrophil extracellular trap (NET) degradation. The method comprises administering a therapeutically effective amount of the DIL3 enzyme or composition described herein. Exemplary indications where a subject is in need of extracellular chromatin degradation (including ET or NET degradation) are disclosed in PCT/US18/47084, the disclosure of which is hereby incorporated by reference.

Neutrophils, the predominant leukocytes in acute inflammation, generate neutrophil extracellular traps (NETs), lattices of high-molecular weight chromatin filaments decorated with biologically active proteins and peptides, which immobilize bacteria in wounds. Systemic accumulation of NETs harms tissues and organs due to their cytotoxic, proinflammatory, and prothrombotic activity. Indeed, NETs are frequently associated with inflammatory, ischemic, and autoimmune conditions, including Systemic Lupus Erythematosus (SLE).

In embodiments, the present invention provides a method for treating, preventing, or managing diseases or conditions characterized by the presence or accumulation of NETs. See Jiménez-Alcázar et al., “Host DNases prevent vascular occlusion by neutrophil extracellular traps.” Science 358(6367): 1202-1206 (2017). A number of stimuli, which sometimes contribute to inflammation and/or pathogenesis, induce NETs. These stimuli include phorbol 12-myristate 13-acetate (PMA), a potent mitogen, lipopolysaccharides (LPS), calcium ionophore A23187, the antibiotic nigericin, which also acts as a potassium ionophore, fungi like Candida albicans, and bacteria like Streptococcus agalactiae (a Group B Streptococcus), Klebsiella pneumoniae and viruses like SARS-CoV2. Leppkes et al. “Vascular occlusion by neutrophil extracellular traps in COVID-19.” EBioMedicine 58 (2020) 102925 (2020); Claushuis et al., “Role of peptidylargininedeiminase 4 in neutrophil extracellular trap formation and host defense during Klebsiella pneumoniae-induced pneumonia-derived sepsis.” J Immunol. 201:1241-1252 (2018); and Kenny et al., “Diverse stimuli engage different neutrophil extracellular trap pathways.” Elife. 6: e24437 (2017). The diseases or conditions characterized by the presence or accumulation of NETs include, but are not limited to, diseases associated with chronic neutrophilia, neutrophil aggregation and/or leukostasis, thrombosis and vascular occlusion, ischemia-reperfusion injury, surgical and traumatic tissue injury, an acute or chronic inflammatory reaction or disease, an autoimmune disease, cardiovascular disease, metabolic disease, systemic inflammation, inflammatory diseases of the respiratory tract, renal inflammatory diseases, inflammatory diseases related to transplanted tissue or hematopoietic stem cell transplantation (e.g. graft-versus-host disease), inflammation caused by viral infections (e.g. COVID-19), and cancer (including leukemia). In embodiments, the present invention provides a method for treating complete or partial vascular or ductal occlusions involving extracellular chromatin, and including NETs In embodiments.

In embodiments, the method comprising administering the compositions described herein to the subject. In embodiments, the subject is at risk of vascular occlusion involving extracellular chromatin, including chromatin released by cancer cells and injured endothelial cells, among others. Thus, in exemplary embodiments, the subject has cancer (e.g., leukemia or solid tumor). In embodiments, the subject has a hematological cancer selected from multiple myeloma (MM), Hodgkin lymphoma (HL), non-Hodgkin lymphoma (NHL), chronic lymphocytic leukemia (CLL), and acute lymphoblastic leukemia (ALL). In embodiments, the subject has metastatic cancer.

Subjects receiving therapy for cancer (including but not limited to T cell therapies) are at risk of tumor lysis syndrome and/or cytokine release syndrome, which occurs when tumor cells release their contents (including chromatin) into the bloodstream. Tumor lysis syndrome is a complication during the treatment of cancer, where large amounts of tumor cells are killed at the same time by cancer treatment. Tumor lysis syndrome and/or cytokine release syndrome occurs commonly after the treatment of lymphomas and leukemias. In embodiments, the therapy described herein treats, reduces, or prevents tumor lysis syndrome.

In still other embodiments, the subject has an inflammatory disease of the respiratory tract, such as the lower respiratory tract. Exemplary diseases include bacterial and viral infections. In embodiments, the subject has acute respiratory distress syndrome (ARDS), Acute Lung Injury (ALI), pneumonia, or asthma. Exemplary viral infections in RSV and coronavirus infection (such as SARS, or SARS-CoV-2, e.g., COVID-19 as well as variants thereof).

In still other embodiments, the subject has a disease or condition other than cancer. In embodiments, the disease or condition is an autoimmune or immunological condition, such as those selected from systemic lupus erythematosus (SLE), rheumatoid arthritis, psoriasis, inflammatory bowel disease, celiac sprue, pernicious anemia, scleroderma, Graves' disease, Sjogren syndrome, autoimmune hemolytic anemia (AIHA), myasthenia gravis, cryoglobulinemia, thrombotic thrombocytopenia purpura (TTP), allograft rejection (e.g., transplant rejection of lung, kidney, heart, intestine, liver, pancreas, etc.), pemphigus vulgaris, vitiligo, Hashimoto's disease, Addison's disease, reactive arthritis, and type 1 diabetes.

In embodiments, the subject has SLE. The discovery of NETs raised the speculation that neutrophils may be the predominant source of autoantigens (i.e. dsDNA, chromatin) in SLE (Brinkmann, et al. Neutrophil Extracellular Traps Kill Bacteria. Science, 303(5663): 1532-1545 (2004)). Indeed, autoantibodies such as anti-dsDNA, anti-histone, and anti-nucleosome antibodies bind to NETs, forming pathological ICs. See, for example, Hakkim, et al., Impairment of neutrophil extracellular trap degradation is associated with lupus nephritis, Proceedings of the National Academy of Sciences 107:9813-9818 (2010). The accumulation of NET-IC breaks immune tolerance via activation of adaptive immune cells that lead to the production of autoantibodies against NET components, forming a vicious cycle of inflammation and autoimmunity. See, for example, Gupta and Kaplan, The role of neutrophils and NETosis in autoimmune and renal diseases. Nat Rev Nephrol. 12(7): 402-13 (2016). Therefore, reducing accumulation of NETs can break the cycle and thus provide an attractive therapeutic strategy for SLE.

In embodiments, the present invention pertains to the treatment of diseases or conditions characterized by deficiency of DIL3, or a deficiency of D1. In some cases, the subject has a mutation (e.g., a loss of function mutation) in a Dnase113 gene or a Dnase1 gene. Such subjects can manifest with an autoimmune disease, such as: systemic lupus erythematosus (SLE), lupus nephritis, scleroderma or systemic sclerosis, rheumatoid arthritis, inflammatory bowel disease, Crohn's disease, ulcerative colitis, and urticarial vasculitis. In some cases, the subject has an acquired inhibitor of D1 (e.g., anti-DNase1-antibody and actin) and/or DIL3 (e.g., anti-Dnase 113-antibody). Such subjects can also have an autoimmune or inflammatory disease (e.g., SLE, systemic sclerosis). In some embodiments, the subject is treated with a D1L3 variant having a half-life extending moiety.

In embodiments, the subject has or is at risk of NETs occluding ductal systems. For example, the D1L3 enzymes or compositions disclosed herein can be administered to a subject to treat pancreatitis, cholangitis, conjunctivitis, mastitis, dry eye disease, Stevens-Johnson syndrome, obstructions of vas deferens, or renal diseases. For example, in embodiments, the D1L3 variant without any half-life extending moiety is administered in eye drop form to a subject having dry eye disease.

In embodiments, the subject has or is at risk of NETs accumulating on endothelial surfaces (e.g. surgical adhesions), the skin (e.g. wounds/scarring), or in synovial joints (e.g. gout and arthritis, e.g., rheumatoid arthritis). The DIL3 enzymes and compositions described herein can be administered to a subject to treat a condition characterized by an accumulation of NETs on an endothelial surface such as, but not limited to, a surgical adhesion.

Other diseases and conditions associated with NETs, which the DIL3 enzymes or compositions disclosed herein may be used to treat or prevent, include: ANCA-associated vasculitis, asthma, chronic obstructive pulmonary disease, a neutrophilic dermatosis, dermatomyositis, burns, cellulitis, meningitis, encephalitis, otitis media, pharyngitis, tonsillitis, pneumonia, endocarditis, cystitis, pyelonephritis, appendicitis, cholecystitis, pancreatitis, uveitis, keratitis, disseminated intravascular coagulation, acute kidney injury, acute respiratory distress syndrome, shock liver, hepatorenal syndrome, myocardial infarction, stroke, ischemic bowel, limb ischemia, testicular torsion, preeclampsia, eclampsia, and solid organ transplant (e.g., kidney, heart, liver, and/or lung transplant). Furthermore, the D1L3 enzymes or compositions disclosed herein can be used to prevent a scar or contracture, e.g., by local application to skin, in an individual at risk thereof, e.g., an individual with a surgical incision, laceration, or burn.

In embodiments, a DIL3 variant of this disclosure (e.g., without a half-life extending moiety) is administered to a subject suffering from or at risk of ischemic stroke. In embodiments, the subject may further undergo therapy with tissue plasminogen activator (tPA).

In embodiments, the subject has a disease that is or has been treated with wild-type DNases, including D1 and streptodornase. Such diseases or conditions include thrombosis, stroke, sepsis, lung injury, atherosclerosis, viral infection, sickle cell disease, myocardial infarction, ear infection, wound healing, liver injury, endocarditis, liver infection, pancreatitis, primary graft dysfunction, limb ischemia reperfusion, kidney injury, blood clotting, alum-induced inflammation, hepatorenal injury, pleural exudations, hemothorax, intrabiliary blood clots, post pneumatic anemia, ulcers, otolaryngological conditions, oral infections, minor injuries, sinusitis, post-operative rhinoplasties, infertility, bladder catheter, wound cleaning, skin reaction test, pneumococcal meningitis, gout, leg ulcers, cystic fibrosis, Kartagener's syndrome, asthma, lobar atelectasis, chronic bronchitis, bronchiectasis, lupus, primary ciliary dyskinesia, bronchiolitis, empyema, pleural infections, cancer, dry eyes disease, lower respiratory tract infections, chronic hematomas, Alzheimer's disease, and obstructive pulmonary disease.

In embodiments, the subject has a loss of function mutation in one or both DIL3 genes, and may exhibit symptoms of SLE, or may be further diagnosed with clinical SLE. In embodiments, the composition is administered no more than about weekly, or no more than about every two or three weeks, or no more than about monthly.

In some aspects, the present disclosure provides an expression construct for improving processing of a polypeptide precursor in a host, the expression construct comprising a signal peptide fused to the polypeptide via a linker. In some embodiments, the linker has at least three amino acids length. In some embodiments, the signal peptide is completely removed from the polypeptide. In some embodiments, the signal peptide is not removed from the polypeptide. In some embodiments, the signal peptide is incompletely processed in the host in the absence of the linker.

In some embodiments, the polypeptide is or comprises an enzyme, a cytokine, a cytokine agonist, a cytokine antagonist, a hormone, a hormone agonist, a hormone antagonist, an antibody or an antigen binding fragment thereof, an antibody-like molecule or an antigen binding fragment thereof, antigen, a component of a vaccine, a fusion protein or a combination thereof. In some embodiments, the enzyme is selected from DNASE1 (D1), DNASE1-LIKE 1 (DIL1), DNASE1-LIKE 2 (DIL2), DNASE1-LIKE 3 Isoform 1 (D1L3), DNASE1-LIKE 3 Isoform 2 (DIL3-2), DNASE2A (D2A), and DNASE2B (D2B), or a variant thereof. In some embodiments, the cytokine is selected from IL-1, IL-2, IL-5, IL-6, IL-10 and IL-13, IL-12, CXCL8 (formerly IL-18), interferon-γ (IFN-γ) and tumor necrosis factor-β (TNF-β), TNF-α, G-CSF, and GM-CSF.

In some embodiments, the hormone is selected from adrenocorticotropic hormone (ACTH), adropin, amylin, angiotensin, atrial natriuretic peptide (ANP), calcitonin, cholecystokinin (CCK), exenatide, gastrin, ghrelin, glucagon, GLP-1, growth hormone, GIP, EPO, follicle-stimulating hormone (FSH), insulin, leptin, luteinizing hormone (LH), melanocyte-stimulating hormone (MSH), oxytocin, parathyroid hormone (PTH), prolactin, renin, somatostatin, thyroid-stimulating hormone (TSH), thyrotropin-releasing hormone (TRH), vasopressin, and vasoactive intestinal peptide (VIP). In some embodiments, the hormone or polypeptide contains a half-life extending moiety, such as an albumin or Fc fusion as described herein. In some embodiments, such fusion is at the C-terminus.

In some embodiments, the linker is at least three, or at least four, or at least five, or at least six, or at least seven, or at least eight, or at least nine, or at least ten, or at least 11, or at least 12, or at least 13, or at least 14, or at least 15, or more amino acid long. In some embodiments, the first amino acid residue of the N-terminal extension is not a Gly residue. In some embodiments, the last amino acid residue of the N-terminal extension is not a Ser residue. In some embodiments, the last amino acid residue of the N-terminal extension is not a polar or charged amino acid residue selected from Ser, Thr, Gln, Asn, Glu, Asp, Arg, His and Lys. In some embodiments, the last amino acid residue of the N-terminal extension is an amino acid selected from Gly, Ala and Val. In some embodiments, the last amino acid residue of the N-terminal extension is a Gly residue. In some embodiments, the first amino acid residue of the N-terminal extension not a Gly residue, and the last amino acid residue of the N-terminal extension is a Gly residue. In some embodiments, the N-terminal extension is predominately Ser and Gly residues, consists essentially of Ser and Gly residues, or consists of Ser and Gly residues. In some embodiments, the linker comprises glycine, cysteine and serine residues. In some embodiments, the linker comprises an amino acid sequence of SGGGG (SEQ ID NO: 60). In some embodiments, the linker comprises an amino acid sequence of CGGGG (SEQ ID NO: 74). In some embodiments, the linker comprises an amino acid sequence of SGGSGGSGG (SEQ ID NO: 61). In some embodiments, the linker comprises an amino acid sequence of SGGSGGSGGSGGSGGSGG (SEQ ID NO: 62). In some embodiments, the linker comprises an amino acid sequence of LVPRG (SEQ ID NO: 64). In some embodiments, the linker comprises an amino acid sequence of SGGGGLVPRGSGGGG (SEQ ID NO: 65).

In some embodiments, the signal peptide is a signal peptide of a natural secretary protein. In some embodiments, the signal peptide is chimeric or synthetic signal peptide that enables protein secretion. In some embodiments, the signal peptide is a prokaryotic signal peptide. In some embodiments, the signal peptide is a microbial signal peptide. In some embodiments, the signal peptide is a eukaryotic signal peptide. In some embodiments, the signal peptide is a yeast signal peptide. In some embodiments, the signal peptide is a mammalian signal peptide. Signal peptides suitable for secretion are disclosed in U.S. Pat. Nos. 5,580,758; 6,107,057; 7,741,075; 10,435,694; 11,306,127; 11,370,815; US Patent Application Publication Nos. 2007/0117186, 2010/0055125, 2016/0168198, the disclosure of each of which is hereby incorporated by reference.

In some embodiments, the N-terminal extension and or the junction of the N-terminal extension and sequence originating from the polypeptide is non-immunogenic. In some embodiments, the last amino acid of the N-terminal extension is not a polar or charged or aromatic amino acid. In some embodiments, the last two, or last three or last four amino acid of the N-terminal extension are not polar, charged and/or aromatic amino acid. In some embodiments, the N-terminal residue is not methionine (Met). In some embodiments, the last amino acid of the N-terminal extension is Gly or Ala. In some embodiments, the N-terminal extension comprises an amino acid having a chemical group suitable for chemical conjugation, the chemical group being selected from a thiol group, amino group, amido group, and carboxyl group. In some embodiments, the chemical modification is site-specific PEGylation, glycosylation, etc. In some embodiments, the signal peptide is selected from DNASE1L3 (SEQ ID NO: 37), Alpha Mating Factor (SEQ ID NO: 38), Alpha Mating Factor Pre-Sequence (SEQ ID NO: 39), Human Serum Albumin (SEQ ID NO: 40), Bovine DNASE1 (SEQ ID NO: 41), Bovine DNASE1+Kex2-Site (SEQ ID NO: 42), Alpha Amylase (SEQ ID NO: 43), Glucoamylase Signal Peptide (SEQ ID NO: 44), Inulinase (SEQ ID NO: 45), Invertase (SEQ ID NO: 46), Killer protein (SEQ ID NO: 47), and Lysozyme (SEQ ID NO: 48).

In some embodiments, the signal peptide is selected from E. coli OmpA signal peptide (SEQ ID NO: 56), E. coli DsbA signal peptide (SEQ ID NO: 67), E. coli ST-II signal peptide (SEQ ID NO: 68), E. coli FimD signal peptide (SEQ ID NO: 55), Salmonella enterica DsbA signal peptide (SEQ ID NO: 51), a synthetic Bordetella pertussis signal peptide (SEQ ID NO: 57), synthetic signal peptide sequences (e.g., SEQ ID NOs: 49, 50, 52, 53 and 54). In some embodiments, the host is a yeast, or a cell line selected from a mammalian cell line, and insect cell line. In some embodiments, the host is Pichia pastoris. In some embodiments, the signal peptide is alpha-mating factor (αMF) pre-pro secretion leader from Saccharomyces cerevisiae (SEQ ID NO: 38).

Other aspects and embodiments of the invention will be apparent from the following examples.

### EXAMPLES

### Example 1. Optimization of N-Terminal Secretion Signal Peptide

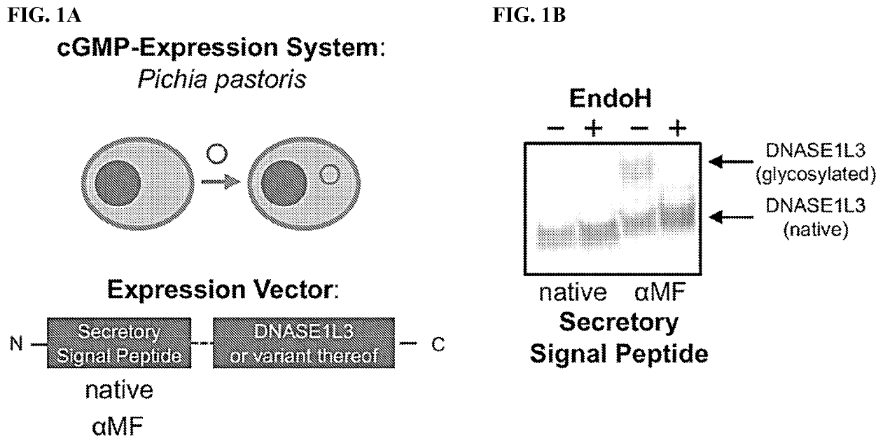

D1L3 enzyme and DIL3-albumin fusion protein expression in Pichia pastoris was disclosed in PCT International Application Publication No. WO2020076817, which is hereby incorporated by reference in its entirety. Briefly, alpha-mating factor (αMF) pre-pro secretion leader from Saccharomyces cerevisiae (SEQ ID NO: 38) was used as an N-terminal secretion signal peptide. See FIG. 1A. aMF is a common and potent tool for heterologous protein expression in Pichia pastoris. As shown in FIG. 1B, the combination of aMF with human D1L3 caused the unexpected non-processing of aMF with concomitant glycosylation.

In FIG. 1B, D1L3 was properly processed, when the N-terminus was led by native secretion signal peptide of DIL3. However, the native signal peptide of DIL3 led to a 3.5-fold decreased expression titer, when compared to aMF.

A series of secretion signal peptides were screened for expression in Pichia pastoris, including the secretion signal peptides of serum albumin, alpha amylase, glycoamylase, inulinase, invertase, killer protein, lysozyme, and bovine DNASE1. In addition, a variant of the bovine DNASE1 secretion signal peptide was tested, which contains an Kex2 cleavage site. See U.S. Pat. No. 7,118,901, which is hereby incorporated by reference in its entirety. The experiments also include the signal peptide of human DNASE1L3 as well two version of the alpha-mating factor (αMF) pre-pro secretion leader from Saccharomyces cerevisiae.

In brief, plasmids were synthesized with cDNA, which coded for various signal peptides of SEQ ID NOs: 37 to 48 at the N-terminus of human DNASE1L3. The plasmids were transformed via electroporation into Pichia pastoris cells. Clonal cells were cultured, and supernatants were analyzed by microfluidic capillary electrophoresis (mCE) to characterize the target protein expression. As shown in Table 1, no or only low levels of DNASE1L3 expression was detected with all secretion signal peptides tested. The best titers were observed with aMF (SEQ ID NO: 38), while DNASE1L3 levels were undetectable with SEQ ID NOs: 41 and 43-46.

TABLE 1
Titer of DNASE1L3 or DNASE1L3-BDD with various secretion signals.

| | DNASE1L3 | DNASE1L3-BDD |
|---|---|---|
| Signal Peptide Identity and SEQ ID NO | (SEQ ID NO: 4) | (SEQ ID NO: 13) |
| DNASE1L3 (SEQ ID NO: 37) | 2 mg/L | 4 mg/L |
| Alpha Mating Factor (SEQ ID NO: 38) | 9 mg/L | 5 mg/L |
| Alpha Mating Factor Pre-Sequence (SEQ ID | 1 mg/L | 3 mg/L |
| NO: 39) | | |
| Human Serum Albumin (SEQ ID NO: 40) | 2 mg/L | 2 mg/L |
| Bovine DNASE1 (SEQ ID NO: 41) | 4 mg/L | 0 mg/L |
| Bovine DNASE1 + Kex2-Site (SEQ ID NO: | 3 mg/L | 2 mg/L |
| 42) | | |
| Alpha Amylase (SEQ ID NO: 43) | N/A | 0 mg/L |
| Glucoamylase Signal Peptide (SEQ ID NO: 44) | N/A | 0 mg/L |
| Inulinase (SEQ ID NO: 45) | N/A | 0 mg/L |
| Invertase (SEQ ID NO: 46) | N/A | 0 mg/L |
| Killer protein (SEQ ID NO: 47) | N/A | 2 mg/L |
| Lysozyme (SEQ ID NO: 48) | N/A | 4 mg/L |

Structural analysis of DNASE1L3 suggests that reduced access of the alpha-mating factor cleaving enzyme Kex2 to its consensus cleavage sequence “LEKR” impairs correct processing into mature DNASE1L3.

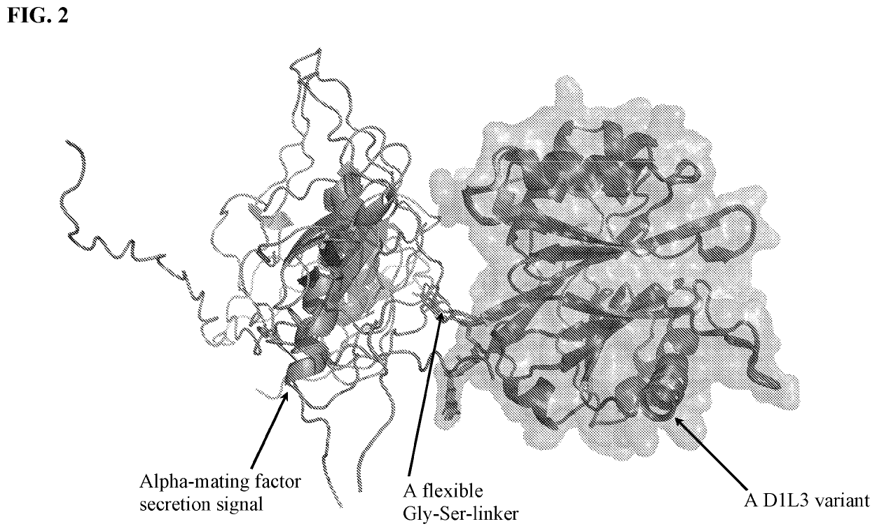

Novel secretion signal peptides were designed that comprise the alpha-mating factor (αMF) pre-pro secretion leader from Saccharomyces cerevisiae (SEQ ID NO: 38) and a linker sequence to facilitate access to the Kex2-cleavage site (FIG. 2).

In pilot studies, flexible glycine-serine linker compositions were tested varying from 1 to 15 amino acids in length. The alpha-mating factor (αMF) pre-pro secretion leader from Saccharomyces cerevisiae (SEQ ID NO: 38)-linker sequences were coupled to DNASE1L3 variant having a basic domain deletion. As shown in Table 2, a strong increase in expression titer was observed starting at a linker length of 5 amino acids.

TABLE 2
Titer of DNASE1L3-BDD with aMF secretion
signal with or without a linker
separating the linker from DNASEIL3-BDD.

| | Expression |
|---|---|
| Secretion signal Peptide + linkers | Titer |
| Alpha Mating Factor (SEQ ID NO: 38) | 5 mg/L |
| without a linker | |
| Alpha Mating Factor + a linker of | 6 mg/L |
| 1 amino acid length | |
| (S) | |
| Alpha Mating Factor + a linker of | 19 mg/L |
| 5 amino acid length | |
| (GGGGS; SEQ ID NO: 58) | |
| Alpha Mating Factor + a linker of | 25 mg/L |
| 15 amino acid length | |
| (GGGGSGGGGSGGGGS; SEQ ID NO: 59) | |

The DNASE1L3 was purified from culture supernatants using affinity chromatography. The analysis by mass spectrometry shows the complete processing of the alpha-mating factor (αMF) pre-pro secretion leader from Saccharomyces cerevisiae (SEQ ID NO: 38) and confirms the predicted N-terminal amino acids sequence.

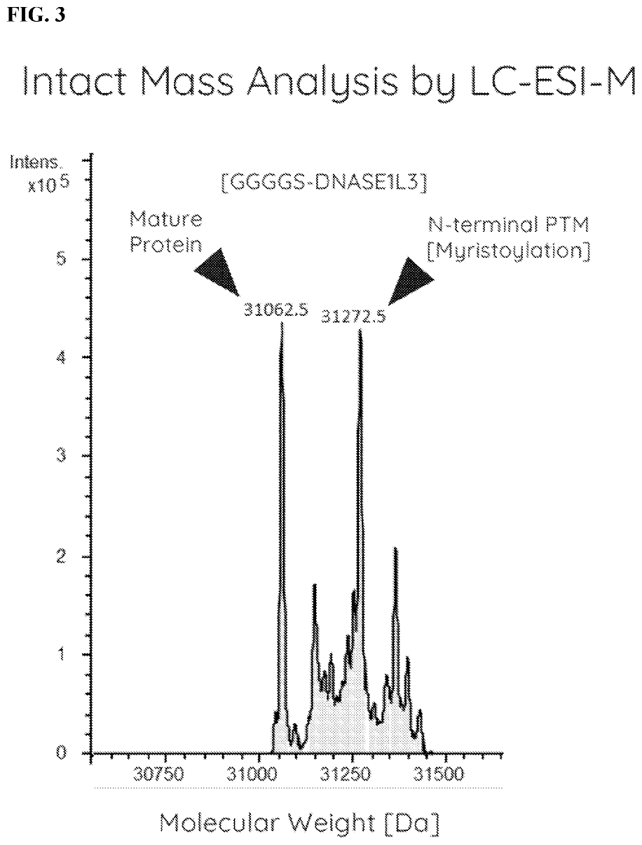

Unexpectedly, as shown in FIG. 3, a mass shift of 210 Da was observed, which likely results from myristoylation of the N-terminal glycine reside. A new set of linker sequences was designed that feature a N-terminal serine residue. The linker length ranged from 1 to 18 amino acids. As shown in Table 3, a strong increase in expression titer was observed starting at a linker length of greater than 3 amino acids.

TABLE 3
Titer of DNASE1L3-BDD with aMF secretion
signal with additional linkers.

| | Expression |
|---|---|
| Secretion signal Peptide | Titer |
| Alpha Mating Factor | 5 mg/L |
| (SEQ ID NO: 38) | |
| Alpha Mating Factor + a linker | 6 mg/L |
| of 1 amino acid length (S) | |
| Alpha Mating Factor + a linker | 25 mg/L |
| of 3 amino acid length (SGG) | |
| Alpha Mating Factor + a linker | 19 mg/L |
| of 5 amino acid length | |
| (SGGGG; SEQ ID NO: 60) | |
| Alpha Mating Factor + a linker | 32 mg/L |
| of 9 amino acid length | |
| (SGGSGGSGG; SEQ ID NO: 61) | |
| Alpha Mating Factor + a linker | 24 mg/L |
| of 18 amino acid length | |
| (SGGSGGSGGSGGSGGSGG; SEQ ID NO: 62) | |

As shown in Table 3, a strong increase in expression titers was observed starting at 3 amino acids. However, mCE and Western Blot analysis suggested that a linker length of 3 amino acids resulted in incomplete processing of the alpha-mating factor (αMF) pre-pro secretion leader from Saccharomyces cerevisiae (SEQ ID NO: 38). Use of a 5-amino acid long linker having the sequence SGGGG (SEQ ID NO: 60) resulted both in increased expression levels and complete processing of the alpha-mating factor (αMF) pre-pro secretion leader from Saccharomyces cerevisiae (SEQ ID NO: 38). All linkers longer than 5 amino acids in length enabled both increased expression levels and complete processing of the alpha-mating factor (αMF) pre-pro secretion leader, including linkers having the sequences SGGSGGSGG (SEQ ID NO: 61), SGGSGGSGSS (SEQ ID NO: 69), and SGGSGGSGGSGGSGGSGG (SEQ ID NO: 62).

Potential immunogenicity of the linkers themselves and the junctions of linkers with DIL3 (e.g., SGGSGGSGSS-MRICSFNVRS (SEQ ID NO: 79); SGGGG-MRICSFNVRS (SEQ ID NO: 80); and SGGSGGSGG-MRICSFNVRS (SEQ ID NO: 81)) was analyzed using an in silico immunogenicity risk prediction algorithm. This analysis indicated that the Ser residue of the SEQ ID NO: 79 junction contributed to a potential dominant epitope that is indicated by underlined-boldface font. Other linkers having a Gly residue at the end did not create a similar dominant epitope. These results indicate, inter alia, that the linker for expression of DIL3 should not end in a Ser residue, and probably other polar residues; and that the linker for expression of DIL3 may end in a Gly residue to reduce immunogenicity risk.

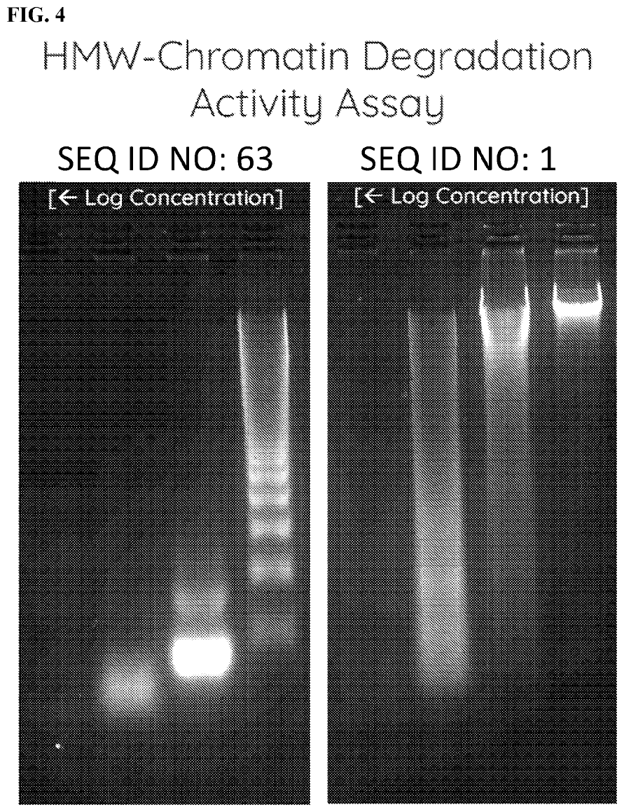

The BDD_DIL3 enzyme (S283_S305del, SEQ ID NO: 13) was produced by the construct comprising Alpha Mating Factor+a SGGGG linker (SEQ ID NO: 60), producing SEQ ID NO: 63, which was analyzed for enzymatic activity on chromatin substrate. Briefly, DNASE 1 (D1) and SEQ ID NO: 63 were produced in Pichia pastoris. Enzymatic activity in culture supernatants was characterized using the degradation of high-molecular weight (HMW)-chromatin (i.e., purified nuclei from HEK293 cells) as a readout. In brief, HMW-chromatin was incubated with equal amounts of D1 or SEQ ID NO: 63. Following incubation, DNA was isolated and visualized via agarose gel electrophoresis (AGE). As shown in FIG. 4, it was observed that, unlike D1, D1L3 S283_S305del with N-terminal SGGGG degrades HMW-chromatin (application Ser. No. 50/300,000,000 base pairs) specifically and efficiently into nucleosomes (app. 180 base pairs), the basic units of chromatin fibers, whereas no such effect was observed in samples with D1. These results suggest that the N-terminal extension does not affect the enzymatic activity of D1L3 enzyme.

### Example 2. Optimization of C-Terminal End

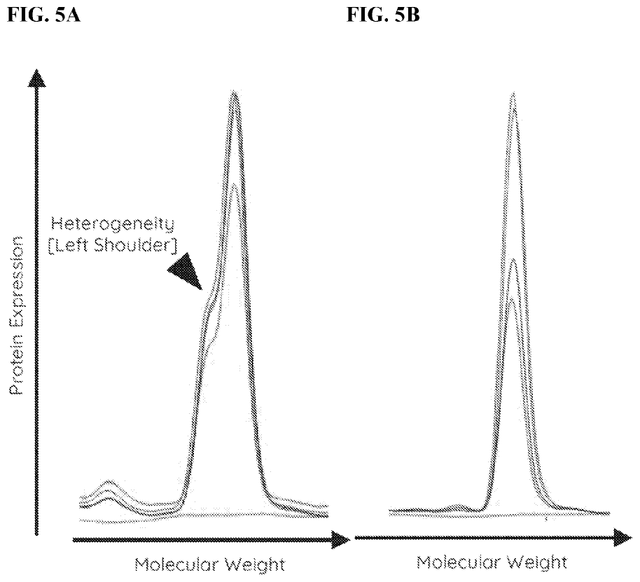

SEQ ID NO: 63 was analyzed by mass spectrometry. As shown in FIG. 5A, the resultant protein elution chromatogram was found to have a left shoulder to the main elution peak, indicating sample heterogeneity. Various derivatives of SEQ ID NO: 63 were analyzed to address sample heterogeneity and it was found that a derivative having the S283_S305delinsSSR mutation (i.e., having a modified C-terminus including a basic domain deletion but featuring the C-terminal addition of 3 amino acids (SSR; SEQ ID NO: 66) produced no such sample heterogeneity (FIG. 5B). These results suggest that the C-terminal extension removes the source of the observed heterogeneity. The enzyme comprising N-terminal and/or C-terminal extension does not affect the enzymatic activity of DIL3 enzyme.

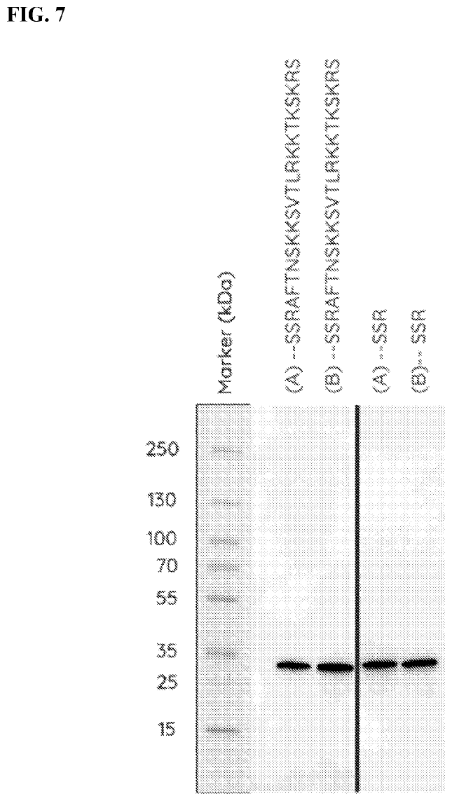

The results were corroborated by studies of two DNASE1L3 variants that feature a wild-type C-terminal amino acid sequence, i.e., SSRAFTNSKKSVTLRKKTKSKRS (SEQ ID NO: 75) or the modified C-terminal amino acid sequence, i.e., SSR, respectively. The variants were expressed in Pichia pastoris using the alpha-mating factor as a signal sequence in conjunction with the N-terminal SGGGG linker (SEQ ID NO: 60). Western Blot analysis of supernatants did not detect the theoretical mass difference of 2.3 kDa between both variants (e.g., as shown in FIG. 7). Furthermore, intact mass analysis showed that the secreted DNASE1L3 variants had an identical mass that matches the theoretical mass of the DNASE1L3 variants with the modified C-terminal amino acid sequence, i.e., SSR. In summary, these data suggest that the wild-type C-terminal amino acid sequence, i.e., SSRAFTNSKKSVTLRKKTKSKRS (SEQ ID NO: 75) is cleaved by Pichia pastoris during the secretion into the modified C-terminal amino acid sequence, i.e., SSR. Pichia pastoris contains processing enzymes, e.g., Kex 1 and Kex2, which have homologues human counterparts, e.g., furin. It is expected that the processing of the DNASE1L3 C-terminus occurs naturally during secretion in humans as well.

### Example 3: Cysteine Mutation

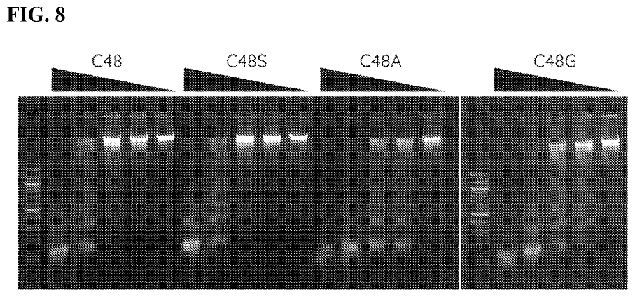

Wild-type D1L3 contains an unpaired cysteine in position 48 (e.g., C48) (with respect to the mature protein sequence). Mutation of this cysteine stabilized the DIL3 and prevents cross-linking to plasma proteins. The impact of different amino acid substitutions at C48 on enzymatic activity was tested. Enzymatic activity was characterized using the degradation of high-molecular weight (HMW)-chromatin (i.e., purified nuclei from HEK293 cells). In brief, HMW-chromatin was incubated with equal amounts of the D1L3 variants. Following incubation, the DNA was isolated and degradation was visualized via agarose gel electrophoresis (AGE). As shown in FIG. 8, mutation of C48A or C48G was associated with an increase in enzymatic activity compared to the DIL3 variant. These results suggest that removal of the unpaired cysteine increases the enzymatic activity of DIL3 enzyme.

### Example 4. Dimerization of DNASE1L3 Via Unpaired Cysteines

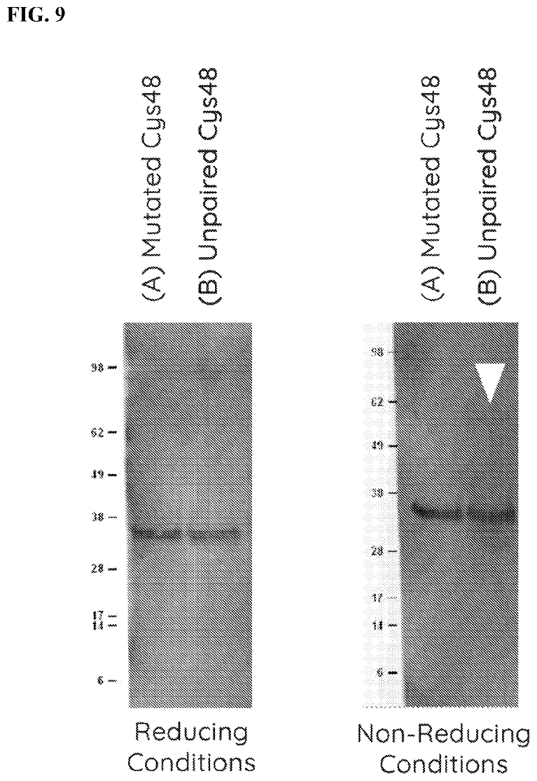

Structural analysis of DNASE1L3 revealed that the unpaired C48 is located on the surface of the molecule. Using Western Blot analysis of Pichia pastoris supernatants, dimerization was observed in DNASE1L3 variants with the unpaired C48, but not in variants with the mutated C48 (e.g., as shown in FIG. 9). These data suggest that dimerization of DNASE1L3 may exist physiologically.

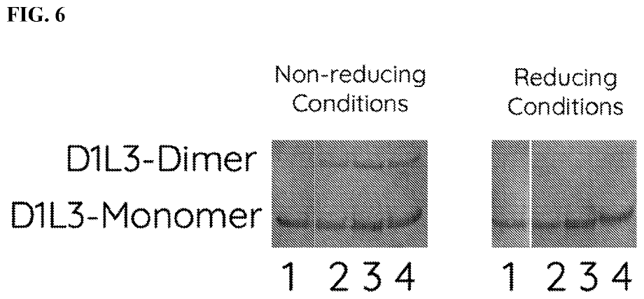

The possibility of inserting a new cysteine in DIL3 variants carrying a mutated C48 was tested to, e.g., enable site-specific PEGylation. The BDD-D1L3 enzyme (A286_S305del) and wild-type D1L3 was produced by a construct comprising Alpha Mating Factor+CGGGG linker (SEQ ID NO: 74). Four samples were compared. Sample 1 (SEQ ID NO: 70) was produced by the construct comprising Alpha Mating Factor+a SGGGG linker, and was a DIL3 variant having a C48A substitution and a C-terminal extension having the sequence SSR. Samples 2-4 (SEQ ID NOs: 71 to 73, respectively) were produced by the construct comprising Alpha Mating Factor+CGGGG linker (SEQ ID NO: 74), and were the D1L3 variants having a C48A/S substitution and having C-terminal extensions. Dimers were detected in DNASE1L3 variants containing a N-terminal cysteine. Analysis of supernatants with an anti-DNASE1L3 Western blot under non-reducing conditions revealed dimerized BDD-D1L3 variants in addition to DIL3 monomers (e.g., as shown in FIG. 6). No dimers were observed under reducing conditions or with a construct comprising Alpha Mating Factor+SGGGG linker. The data confirm that the introduction of the CGGGG linker enables the dimerization via disulfide bridge of the N-terminal cysteine residue. Similarly, the N-terminal variants, e.g., to a cysteine residue, allows for increased functionality, e.g., a site-specific PEGylation.

### SEQUENCES

Wild-Type Human DNASES
DNASEI (NP_005212.2): Signal Peptide, Mature Protein:
SEQ ID NO: 1
MRGMKLLGALLALAALLQGAVSLKIAAFNIQTFGETKMSNATLVSYIVQILSRYDIALVQ
EVRDSHLTAVGKLLDNLNQDAPDTYHYVVSEPLGRNSYKERYLFVYRPDQVSAVDSYYYD
DGCEPCGNDTFNREPAIVRFFSRFTEVREFAIVPLHAAPGDAVAEIDALYDVYLDVQEKW
GLEDVMLMGDFNAGCSYVRPSQWSSIRLWTSPTFQWLIPDSADTTATPTHCAYDRIVVAG
MLLRGAVVPDSALPENFQAAYGLSDQLAQAISDHYPVEVMLK
DNASE1-LIKE 1 (NP_006721.1): Signal Peptide; Mature Protein:
SEQ ID NO: 2
MHYPTALLFLILANGAQAFRICAFNAQRLTLAKVAREQVMDTLVRILARCDIMVLQEVVD
SSGSAIPLLLRELNREDGSGPYSTLSSPQLGRSTYMETYVYFYRSHKTQVLSSYVYNDED
DVFAREPFVAQFSLPSNVLPSLVLVPLHTTPKAVEKELNALYDVFLEVSQHWQSKDVILL
GDFNADCASLTKKRLDKLELRTEPGFHWVIADGEDTTVRASTHCTYDRVVLHGERCRSLL
HTAAAFDFPTSFQLTEEEALNISDHYPVEVELKLSQAHSVQPLSLTVLLLLSLLSPQLCP
AA
DNASE1-LIKE 2 (NP_001365.1): Signal Peptide, Mature Protein:
SEQ ID NO: 3
MGGPRALLAALWALEAAGTAALRIGAFNIQSFGDSKVSDPACGSIIAKILAGYDLALVQE
VRDPDLSAVSALMEQINSVSEHEYSFVSSQPLGRDQYKEMYLFVYRKDAVSVVDTYLYPD
PEDVFSREPFVVKFSAPGTGERAPPLPSRRALTPPPLPAAAQNLVLIPLHAAPHQAVAEI
DALYDVYLDVIDKWGTDDMLFLGDFNADCSYVRAQDWAAIRLRSSEVFKWLIPDSADTTV
GNSDCAYDRIVACGARLRRSLKPQSATVHDFQEEFGLDQTQALAISDHFPVEVTLKFHR
DNASE1-LIKE 3; Isoform 1 (NP_004935.1): Signal Peptide, Mature Protein:
SEQ ID NO: 4
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDEVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQSSRAFTNSKKSVTLRKKT
KSKRS
DNASEI-LIKE 3, Isoform 2 (NP_001243489.1): Signal Peptide; Mature Protein:
SEQ ID NO: 5
MSRELAPLLLLLLSIHSALAMRICSFNVRSFGESKQEDKNAMDVIVKVIKRCDIILVMEI
KDSNNRICPILMEKLNREKLVSVKRSYHYHDYQDGDADVFSREPFVVWFQSPHTAVKDFV
IIPLHTTPETSVKEIDELVEVYTDVKHRWKAENFIEMGDFNAGCSYVPKKAWKNIRLRTD
PRFVWLIGDQEDTTVKKSTNCAYDRIVLRGQEIVSSVVPKSNSVFDFQKAYKLTEEEALD
VSDHFPVEFKLQSSRAFTNSKKSVTLRKKTKSKRS
DNASE2A (000115): Signal Peptide; Mature Protein:
SEQ ID NO: 6
MIPLLLAALLCVPAGALTCYGDSGQPVDWFVVYKLPALRGSGEAAQRGLQYKYLDESSGG
WRDGRALINSPEGAVGRSLQPLYRSNTSQLAFLLYNDQPPQPSKAQDSSMRGHTKGVLLL
DHDGGFWLVHSVPNFPPPASSAAYSWPHSACTYGQTLLCVSFPFAQFSKMGKQLTYTYPW
VYNYQLEGIFAQEFPDLENVVKGHHVSQEPWNSSITLTSQAGAVFQSFAKFSKEGDDLYS
GWLAAALGTNLQVQFWHKTVGILPSNCSDIWQVLNVNQIAFPGPAGPSENSTEDHSKWCV
SPKGPWTCVGDMNRNQGEEQRGGGTLCAQLPALWKAFQPLVKNYQPCNGMARKPSRAYKI
DNASE2B (Q8WZ79): Signal Peptide; Mature Protein:
SEQ ID NO: 7
MKQKMMARLLRTSFALLFLGLFGVLGAATISCRNEEGKAVDWFTFYKLPKRQNKESGETG
LEYLYLDSTTRSWRKSEQLMNDTKSVLGRTLQQLYEAYASKSNNTAYLIYNDGVPKPVNY
SRKYGHTKGLLLWNRVQGFWLIHSIPQFPPIPEEGYDYPPTGRRNGQSGICITFKYNQYE
AIDSQLLVCNPNVYSCSIPATFHQELIHMPQLCTRASSSEIPGRLLTTLQSAQGQKFLHF
AKSDSFLDDIFAAWMAQRLKTHLLTETWQRKRQELPSNCSLPYHVYNIKAIKLSRHSYFS
SYQDHAKWCISQKGTKNRWTCIGDLNRSPHQAFRSGGFICTQNWQIYQAFQGLVLYYESC
K
C-terminal deletion mutants of Human DNASE1L3
S305del: Signal Peptide; Mature Protein:
SEQ ID NO: 8
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDFVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQSSRAFTNSKKSVTLRKKT
KSKR
K303_S305del: Signal Peptide; Mature Protein:
SEQ ID NO: 9
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDEVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQSSRAFTNSKKSVTLRKKT
KS
V294_S305del: Signal Peptide; Mature Protein:
SEQ ID NO: 10
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDEVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQSSRAFTNSKKS
K291 S305del: Signal Peptide; Mature Protein:
SEQ ID NO: 11
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDEVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVEDFQKAYKLTEEEALDVSDHFPVEFKLQSSRAFTNS
R285 S305del: Signal Peptide; Mature Protein:
SEQ ID NO: 12
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDEVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQSS
S283 S305del: Signal Peptide; Mature Protein:
SEQ ID NO: 13
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDEVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQ
K298 S305del: Signal Peptide; Mature Protein:
SEQ ID NO: 14
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDFVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQSSRAFTNSKKSVTLR
R297 S305del: Signal Peptide; Mature Protein:
SEQ ID NO: 15
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDEVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQSSRAFTNSKKSVTL
S293 S305del: Signal Peptide; Mature Protein:
SEQ ID NO: 16
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDEVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQSSRAFTNSKK
K292 S305del: Signal Peptide; Mature Protein:
SEQ ID NO: 17
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDFVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQSSRAFTNSK
Alpha Mating Factor Signal Peptide; a linker; DIL3 S283_S305del Mature Protein (the
enzyme as produced by Pichia pastoris includes the SGGGG linker):
SEQ ID NO: 63
MRFPSIFTAVLFAASSALAAPVNTTTEDETAQIPAEAVIGYSDLEGDFDVAVLPFSNSTN
NGLLFINTTIASIAAKEEGVSLEKRSGGGGMRICSFNVRSFGESKQEDKNAMDVIVKVIK
RCDIILVMEIKDSNNRICPILMEKLNRNSRRGITYNYVISSRLGRNTYKEQYAFLYKEKL
VSVKRSYHYHDYQDGDADVFSREPFVVWFQSPHTAVKDFVIIPLHTTPETSVKEIDELVE
VYTDVKHRWKAENFIFMGDENAGCSYVPKKAWKNIRLRTDPRFVWLIGDQEDTTVKKSTN
CAYDRIVLRGQEIVSSVVPKSNSVEDFQKAYKLTEEEALDVSDHFPVEFKLQ
Alpha Mating Factor Signal Peptide; a linker; D1L3 S283_S305del Mature Protein having
a C48S substitution; C-terminal extension (the enzyme as produced by Pichia pastoris
includes the SGGGG linker and the C-terminal extension):
SEQ ID NO: 66
MRFPSIFTAVLFAASSALAAPVNTTTEDETAQIPAEAVIGYSDLEGDFDVAVLPFSNSTN
NGLLFINTTIASIAAKEEGVSLEKRSGGGGMRICSFNVRSFGESKQEDKNAMDVIVKVIK
RCDIILVMEIKDSNNRISPILMEKLNRNSRRGITYNYVISSRLGRNTYKEQYAFLYKEKL
VSVKRSYHYHDYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQEDTTVKKSTN
CAYDRIVLRGQEIVSSVVPKSNSVFDFQKAYKLTEEEALDVSDHFPVEFKLQSSR
Alpha Mating Factor Signal Peptide; a linker; D1L3 S283_S305del Mature Protein having
a C48A substitution; C-terminal extension (the enzyme as produced by Pichia pastoris
includes the SGGGG linker and the C-terminal extension):
SEQ ID NO: 70
MRFPSIFTAVLFAASSALAAPVNTTTEDETAQIPAEAVIGYSDLEGDFDVAVLPFSNSTN
NGLLFINTTIASIAAKEEGVSLEKRSGGGGMRICSFNVRSFGESKQEDKNAMDVIVKVIK
RCDIILVMEIKDSNNRIAPILMEKLNRNSRRGITYNYVISSRLGRNTYKEQYAFLYKEKL
VSVKRSYHYHDYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQEDTTVKKSTN
CAYDRIVLRGQEIVSSVVPKSNSVFDFQKAYKLTEEEALDVSDHFPVEFKLQSSR
Alpha Mating Factor Signal Peptide; a linker; D1L3 S283_S305del Mature Protein having
a C48A substitution, C-terminal extension (the enzyme as produced by Pichia pastoris
includes the CGGGG linker and the C-terminal extension):
SEQ ID NO: 71
MRFPSIFTAVLFAASSALAAPVNTTTEDETAQIPAEAVIGYSDLEGDFDVAVLPFSNSTN
NGLLFINTTIASIAAKEEGVSLEKRCGGGGMRICSFNVRSFGESKQEDKNAMDVIVKVIK
RCDIILVMEIKDSNNRIAPILMEKLNRNSRRGITYNYVISSRLGRNTYKEQYAFLYKEKL
VSVKRSYHYHDYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQEDTTVKKSTN
CAYDRIVLRGQEIVSSVVPKSNSVFDFQKAYKLTEEEALDVSDHFPVEFKLQSSR
Alpha Mating Factor Signal Peptide; a linker; D1L3 S283_S305del Mature Protein having
a C48S substitution; C-terminal extension (the enzyme as produced by Pichia pastoris
includes the CGGGG linker and the C-terminal extension):
SEQ ID NO: 72
MRFPSIFTAVLFAASSALAAPVNTTTEDETAQIPAEAVIGYSDLEGDFDVAVLPFSNSTN
NGLLFINTTIASIAAKEEGVSLEKRCGGGGMRICSFNVRSFGESKQEDKNAMDVIVKVIK
RCDIILVMEIKDSNNRISPILMEKLNRNSRRGITYNYVISSRLGRNTYKEQYAFLYKEKL
VSVKRSYHYHDYQDGDADVFSREPFVVWFQSPHTAVKDFVIIPLHTTPETSVKEIDELVE
VYTDVKHRWKAENFIFMGDFNAGCSYVPKKAWKNIRLRTDPREVWLIGDQEDTTVKKSTN
CAYDRIVLRGQEIVSSVVPKSNSVFDFQKAYKLTEEEALDVSDHFPVEFKLQSSR
Alpha Mating Factor Signal Peptide;; a linker; a DIL3 variant having a C48A substitution,
C-terminal basic domain (the enzyme as produced by Pichia pastoris includes the CGGGG
linker and the C-terminal extension):
SEQ ID NO: 73
MRFPSIFTAVLFAASSALAAPVNTTTEDETAQIPAEAVIGYSDLEGDFDVAVLPFSNSTN
NGLLFINTTIASIAAKEEGVSLEKRCGGGGMRICSFNVRSFGESKQEDKNAMDVIVKVIK
RCDIILVMEIKDSNNRIAPILMEKLNRNSRRGITYNYVISSRLGRNTYKEQYAFLYKEKL
VSVKRSYHYHDYQDGDADVFSREPFVVWFQSPHTAVKDFVIIPLHTTPETSVKEIDELVE
VYTDVKHRWKAENFIFMGDFNAGCSYVPKKAWKNIRLRTDPRFVWLIGDQEDTTVKKSTN
CAYDRIVLRGQEIVSSVVPKSNSVEDFQKAYKLTEEEALDVSDHFPVEFKLQSSRAFTNS
KKSVTLRKKTKSKRS
N-terminal extension; D1L3 S283_S305del Mature Protein having a C48A substitution; C-
terminal extension (the enzyme as produced by Pichia pastoris includes the SGGGG linker
and the C-terminal extension):
SEQ ID NO: 77
SGGGGMRICSFNVRSFGESKQEDKNAMDVIVKVIKRCDIILVMEIKDSNNRIAPILMEKL
NRNSRRGITYNYVISSRLGRNTYKEQYAFLYKEKLVSVKRSYHYHDYQDGDADVESREPE
VVWFQSPHTAVKDFVIIPLHTTPETSVKEIDELVEVYTDVKHRWKAENFIFMGDENAGCS
YVPKKAWKNIRLRTDPRFVWLIGDQEDTTVKKSTNCAYDRIVLRGQEIVSSVVPKSNSVF
DFQKAYKLTEEEALDVSDHFPVEFKLQSSR

### Linker Sequences

SEQ ID NO: 18
GGGGS
SEQ ID NO: 19
GGGGSGGGGSGGGGS
SEQ ID NO: 20
APAPAPAPAPAPAP
SEQ ID NO: 21
AEAAAKEAAAKA
SEQ ID NO: 22
SGGSGSS
SEQ ID NO: 23
SGGSGGSGGSGGSGSS
SEQ ID NO: 24
SGGSGGSGGSGGSGGSGGSGGSGGSGGSGSS
SEQ ID NO: 25
GGSGGSGGSGGSGGSGGSGGSGGSGGSGS

### Other Sequences

Human Serum Albumin (Mature Protein):
SEQ ID NO: 26
DAHKSEVAHRFKDLGEENFKALVLIAFAQYLQQCPFEDHVKLVNEVTEFAKTCVADESAE
NCDKSLHTLFGDKLCTVATLRETYGEMADCCAKQEPERNECFLQHKDDNPNLPRLVRPEV
DVMCTAFHDNEETFLKKYLYEIARRHPYFYAPELLFFAKRYKAAFTECCQAADKAACLLP
KLDELRDEGKASSAKQRLKCASLQKFGERAFKAWAVARLSQRFPKAEFAEVSKLVTDLTK
VHTECCHGDLLECADDRADLAKYICENQDSISSKLKECCEKPLLEKSHCIAEVENDEMPA
DLPSLAADFVESKDVCKNYAEAKDVFLGMFLYEYARRHPDYSVVLLLRLAKTYETTLEKC
CAAADPHECYAKVEDEFKPLVEEPQNLIKQNCELFEQLGEYKFQNALLVRYTKKVPQVST
PTLVEVSRNLGKVGSKCCKHPEAKRMPCAEDYLSVVLNQLCVLHEKTPVSDRVTKCCTES
LVNRRPCFSALEVDETYVPKEFNAETFTFHADICTLSEKERQIKKQTALVELVKHKPKAT
KEQLKAVMDDFAAFVEKCCKADDKETCFAEEGKKLVAASQAALGL
Human Factor XI:
SEQ ID NO: 27
MIFLYQVVHFILFTSVSGECVTQLLKDTCFEGGDITTVFTPSAKYCQVVCTYHPRCLLFT
FTAESPSEDPTRWFTCVLKDSVTETLPRVNRTAAISGYSFKQCSHQISACNKDIYVDLDM
KGINYNSSVAKSAQECQERCTDDVHCHFFTYATRQFPSLEHRNICLLKHTQTGTPTRITK
LDKVVSGFSLKSCALSNLACIRDIFPNTVFADSNIDSVMAPDAFVCGRICTHHPGCLEFT
FFSQEWPKESQRNLCLLKTSESGLPSTRIKKSKALSGFSLQSCRHSIPVFCHSSFYHDTD
FLGEELDIVAAKSHEACQKLCTNAVRCQFFTYTPAQASCNEGKGKCYLKLSSNGSPTKIL
HGRGGISGYTLRLCKMDNECTTKIKPRIVGGTASVRGEWPWQVTLHTTSPTQRHLCGGSI
IGNQWILTAAHCFYGVESPKILRVYSGILNQSEIKEDTSFFGVQEIIIHDQYKMAESGYD
IALLKLETTVNYTDSQRPICLPSKGDRNVIYTDCWVTGWGYRKLRDKIQNTLQKAKIPLV
TNEECQKRYRGHKITHKMICAGYREGGKDACKGDSGGPLSCKHNEVWHLVGITSWGEGCA
QRERPGVYTNVVEYVDWILEKTQAV
Human prekallikrein:
SEQ ID NO: 28
MILFKQATYFISLFATVSCGCLTQLYENAFFRGGDVASMYTPNAQYCQMRCTFHPRCLLF
SFLPASSINDMEKRFGCFLKDSVTGTLPKVHRTGAVSGHSLKQCGHQISACHRDIYKGVD
MRGVNFNVSKVSSVEECQKRCTNNIRCQFFSYATQTFHKAEYRNNCLLKYSPGGTPTAIK
VLSNVESGFSLKPCALSEIGCHMNIFQHLAFSDVDVARVLTPDAFVCRTICTYHPNCLFF
TFYTNVWKIESQRNVCLLKTSESGTPSSSTPQENTISGYSLLTCKRTLPEPCHSKIYPGV
DFGGEELNVTFVKGVNVCQETCTKMIRCQFFTYSLLPEDCKEEKCKCFLRLSMDGSPTRI
AYGTQGSSGYSLRLCNTGDNSVCTTKTSTRIVGGINSSWGEWPWQVSLQVKLTAQRHLCG
GSLIGHQWVLTAAHCFDGLPLQDVWRIYSGILNLSDITKDTPFSQIKEIIIHQNYKVSEG
NHDIALIKLQAPLNYTEFQKPICLPSKGDTSTIYTNCWVTGWGFSKEKGEIQNILQKVNI
PLVTNEECQKRYQDYKITQRMVCAGYKEGGKDACKGDSGGPLVCKHNGMWRLVGITSWGE
GCARREQPGVYTKVAEYMDWILEKTQSSDGKAQMQSPA

### Activatable Linker Sequences

FXIIa-susceptible linker (Factor XI peptide):
SEQ ID NO: 29
CTTKIKPRIVGGTASVRGEWPWQVT
FXIIa-susceptible linker
SEQ ID NO: 30
GGGGSPRIGGGGS
FXIIa-susceptible linker (Prekallikrein peptide):
SEQ ID NO: 31
VCTTKTSTRIVGGINSSWGEWPWQVS
FXIIa-susceptible linker (Prekallikrein
peptide):
SEQ ID NO: 32
STRIVGG
thrombin-susceptible linker 1:
LVPRG
SEQ ID NO: 64
thrombin-susceptible linker 2:
SEQ ID NO: 65
SGGGGLVPRGSGGGG

### BD-Deleted DIL3 Fusion Proteins

Albumin-DNASE1L3 Variant-Fusion Protein. (Albumin, DNASE1L3 Variant
S283_S305del):
SEQ ID NO: 33
DAHKSEVAHRFKDLGEENFKALVLIAFAQYLQQCPFEDHVKLVNEVTEFAKTCVADESAE
NCDKSLHTLFGDKLCTVATLRETYGEMADCCAKQEPERNECFLQHKDDNPNLPRLVRPEV
DVMCTAFHDNEETFLKKYLYEIARRHPYFYAPELLFFAKRYKAAFTECCQAADKAACLLP
KLDELRDEGKASSAKQRLKCASLQKFGERAFKAWAVARLSQRFPKAEFAEVSKLVTDLTK
VHTECCHGDLLECADDRADLAKYICENQDSISSKLKECCEKPLLEKSHCIAEVENDEMPA
DLPSLAADFVESKDVCKNYAEAKDVFLGMFLYEYARRHPDYSVVLLLRLAKTYETTLEKC
CAAADPHECYAKVEDEFKPLVEEPQNLIKQNCELFEQLGEYKFQNALLVRYTKKVPQVST
PTLVEVSRNLGKVGSKCCKHPEAKRMPCAEDYLSVVLNQLCVLHEKTPVSDRVTKCCTES
LVNRRPCFSALEVDETYVPKEFNAETFTFHADICTLSEKERQIKKQTALVELVKHKPKAT
KEQLKAVMDDFAAFVEKCCKADDKETCFAEEGKKLVAASQAALGLSGGSGGSGGSGGSGG
SGGSGGSGGSGGSGSSMRICSFNVRSFGESKQEDKNAMDVIVKVIKRCDIILVMEIKDSN
NRICPILMEKLNRNSRRGITYNYVISSRLGRNTYKEQYAFLYKEKLVSVKRSYHYHDYQD
GDADVFSREPFVVWFQSPHTAVKDFVIIPLHTTPETSVKEIDELVEVYTDVKHRWKAENF
IFMGDFNAGCSYVPKKAWKNIRLRTDPRFVWLIGDQEDTTVKKSTNCAYDRIVLRGQEIV
SSVVPKSNSVFDFQKAYKLTEEEALDVSDHFPVEFKLQ
BD-Deleted DIL3-Fc Fusion Protein. (Signal Peptide, DNASE1L3
(S283_S305del), Fc Fragment):
SEQ ID NO: 34
MSRELAPLLLLLLSIHSALAMRICSFNVRSFGESKQEDKNAMDVIVKVIKRCDIILVMEI
KDSNNRICPILMEKLNRNSRRGITYNYVISSRLGRNTYKEQYAFLYKEKLVSVKRSYHYH
DYQDGDADVFSREPFVVWFQSPHTAVKDFVIIPLHTTPETSVKEIDELVEVYTDVKHRWK
AENFIFMGDFNAGCSYVPKKAWKNIRLRTDPRFVWLIGDQEDTTVKKSTNCAYDRIVLRG
QEIVSSVVPKSNSVFDFQKAYKLTEEEALDVSDHFPVEFKLQSSGGSGGSGGSGGSGGSG
GSGGSGGSGGSDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPEN
NYKTTPPVLDSDGSFFLYSKLTVDKSRWQQGNVFSVSVMHEALHNHYTQKSLSLSPGK
Albumin-DNASE1L3 Variant-Fusion Protein. (Albumin-linker-DNASE1L3 Variant
S283_S305del having a C48A substitution-C-terminal extension):
SEQ ID NO: 76
DAHKSEVAHRFKDLGEENFKALVLIAFAQYLQQCPFEDHVKLVNEVTEFAKTCVADESAE
NCDKSLHTLFGDKLCTVATLRETYGEMADCCAKQEPERNECFLQHKDDNPNLPRLVRPEV
DVMCTAFHDNEETFLKKYLYEIARRHPYFYAPELLFFAKRYKAAFTECCQAADKAACLLP
KLDELRDEGKASSAKQRLKCASLQKFGERAFKAWAVARLSQRFPKAEFAEVSKLVTDLTK
VHTECCHGDLLECADDRADLAKYICENQDSISSKLKECCEKPLLEKSHCIAEVENDEMPA
DLPSLAADEVESKDVCKNYAEAKDVELGMFLYEYARRHPDYSVVLLLRLAKTYETTLEKC
CAAADPHECYAKVEDEFKPLVEEPQNLIKQNCELFEQLGEYKFQNALLVRYTKKVPQVST
PTLVEVSRNLGKVGSKCCKHPEAKRMPCAEDYLSVVLNQLCVLHEKTPVSDRVTKCCTES
LVNRRPCFSALEVDETYVPKEFNAETFTFHADICTLSEKERQIKKQTALVELVKHKPKAT
KEQLKAVMDDFAAFVEKCCKADDKETCFAEEGKKLVAASQAALGLGGGGSGGGGSGGGGS
GGGGSGGGGSGGGGMRICSFNVRSFGESKQEDKNAMDVIVKVIKRCDIILVMEIKDSNNR
IAPILMEKLNRNSRRGITYNYVISSRLGRNTYKEQYAFLYKEKLVSVKRSYHYHDYQDGD
ADVFSREPFVVWFQSPHTAVKDFVIIPLHTTPETSVKEIDELVEVYTDVKHRWKAENFIF
MGDFNAGCSYVPKKAWKNIRLRTDPRFVWLIGDQEDTTVKKSTNCAYDRIVLRGQEIVSS
VVPKSNSVFDFQKAYKLTEEEALDVSDHFPVEFKLQSSR
Albumin-DNASEIL3 Variant-Fusion Protein. (Albumin-linker-DNASE1L3 Variant
S283_S305del having a C48A substitution-C-terminal extension):
SEQ ID NO: 78
DAHKSEVAHRFKDLGEENFKALVLIAFAQYLQQCPFEDHVKLVNEVTEFAKTCVADESAE
NCDKSLHTLFGDKLCTVATLRETYGEMADCCAKQEPERNECFLQHKDDNPNLPRLVRPEV
DVMCTAFHDNEETFLKKYLYEIARRHPYFYAPELLFFAKRYKAAFTECCQAADKAACLLP
KLDELRDEGKASSAKQRLKCASLQKFGERAFKAWAVARLSQRFPKAEFAEVSKLVTDLTK
VHTECCHGDLLECADDRADLAKYICENQDSISSKLKECCEKPLLEKSHCIAEVENDEMPA
DLPSLAADFVESKDVCKNYAEAKDVFLGMFLYEYARRHPDYSVVLLLRLAKTYETTLEKC
CAAADPHECYAKVFDEFKPLVEEPQNLIKQNCELFEQLGEYKFQNALLVRYTKKVPQVST
PTLVEVSRNLGKVGSKCCKHPEAKRMPCAEDYLSVVLNQLCVLHEKTPVSDRVTKCCTES
LVNRRPCFSALEVDETYVPKEFNAETFTFHADICTLSEKERQIKKQTALVELVKHKPKAT
KEQLKAVMDDFAAFVEKCCKADDKETCFAEEGKKLVAASQAALGL SGGSGGSGGSGGSGG
SGGSGGSGGSGGSGGMRICSFNVRSFGESKQEDKNAMDVIVKVIKRCDIILVMEIKDSNN
RIAPILMEKLNRNSRRGITYNYVISSRLGRNTYKEQYAFLYKEKLVSVKRSYHYHDYQDG
DADVFSREPFVVWFQSPHTAVKDFVIIPLHTTPETSVKEIDELVEVYTDVKHRWKAENFI
FMGDFNAGCSYVPKKAWKNIRLRTDPRFVWLIGDQEDTTVKKSTNCAYDRIVLRGQEIVS
SVVPKSNSVFDFQKAYKLTEEEALDVSDHFPVEFKLQSSR

### Wild-Type D1L3 Fusion Proteins

Albumin-WT DNASEIL3-Fusion Protein.
(Albumin, DNASE1L3):
SEQ ID NO: 35
DAHKSEVAHRFKDLGEENFKALVLIAFAQYLQQCPFEDHVKLVNE
VTEFAKTCVADESAENCDKSLHTLFGDKLCTVATLRETYGEMADC
CAKQEPERNECFLQHKDDNPNLPRLVRPEVDVMCTAFHDNEETFL
KKYLYEIARRHPYFYAPELLFFAKRYKAAFTECCQAADKAACLLP
KLDELRDEGKASSAKQRLKCASLQKFGERAFKAWAVARLSQRFPK
AEFAEVSKLVTDLTKVHTECCHGDLLECADDRADLAKYICENQDS
ISSKLKECCEKPLLEKSHCIAEVENDEMPADLPSLAADEVESKDV
CKNYAEAKDVFLGMFLYEYARRHPDYSVVLLLRLAKTYETTLEKC
CAAADPHECYAKVEDEFKPLVEEPQNLIKQNCELFEQLGEYKFQN
ALLVRYTKKVPQVSTPTLVEVSRNLGKVGSKCCKHPEAKRMPCAE
DYLSVVLNQLCVLHEKTPVSDRVTKCCTESLVNRRPCFSALEVDE
TYVPKEFNAETFTFHADICTLSEKERQIKKQTALVELVKHKPKAT
KEQLKAVMDDFAAFVEKCCKADDKETCFAEEGKKLVAASQAALGL
GGGGSGGGGSGGGGSMRICSFNVRSFGESKQEDKNAMDVIVKVIK
RCDIILVMEIKDSNNRICPILMEKLNRNSRRGITYNYVISSRLGR
NTYKEQYAFLYKEKLVSVKRSYHYHDYQDGDADVFSREPFVVWFQ
SPHTAVKDFVIIPLHTTPETSVKEIDELVEVYTDVKHRWKAENFI
FMGDFNAGCSYVPKKAWKNIRLRTDPRFVWLIGDQEDTTVKKSTN
CAYDRIVLRGQEIVSSVVPKSNSVFDFQKAYKLTEEEALDVSDHF
PVEFKLQSSRAFTNSKKSVTLRKKTKSKRS
WT DNASEIL3-Fc Fusion Protein.
(Signal Peptide, DNASE1L3, Fc Fragment):
SEQ ID NO: 36
MSRELAPLLLLLLSIHSALAMRICSFNVRSFGESKQEDKNAMDVI
VKVIKRCDIILVMEIKDSNNRICPILMEKLNRNSRRGITYNYVIS
SRLGRNTYKEQYAFLYKEKLVSVKRSYHYHDYQDGDADVFSREPF
VVWFQSPHTAVKDFVIIPLHTTPETSVKEIDELVEVYTDVKHRWK
AENFIFMGDFNAGCSYVPKKAWKNIRLRTDPRFVWLIGDQEDTTV
KKSTNCAYDRIVLRGQEIVSSVVPKSNSVFDFQKAYKLTEEEALD
VSDHFPVEFKLQSSRAFTNSKKSVTLRKKTKSKRSSGGSGGSGGS
GGSGGSGGSGGSGGSGGSDKTHTCPPCPAPELLGGPSVFLFPPKP
KDTLMISRTPEVTCVVVDVSHEDPEVKFNWYVDGVEVHNAKTKPR
EEQYNSTYRVVSVLTVLHQDWLNGKEYKCKVSNKALPAPIEKTIS
KAKGQPREPQVYTLPPSRDELTKNQVSLTCLVKGFYPSDIAVEWE
SNGQPENNYKTTPPVLDSDGSFFLYSKLTVDKSRWQQGNVFSVSV
MHEALHNHYTQKSLSLSPGK

### Signal Peptides

DNASEIL3
SEQ ID NO: 37
MSRELAPLLLLLLSIHSALA
Alpha Mating Factor
SEQ ID NO: 38
MRFPSIFTAVLFAASSALAAPVNTTTEDETAQIPAEAVIGYSD
LEGDEDVAVLPFSNSTNNGLLFINTTIASIAAKEEGVSLEKR
SEQ ID NO: 39
Alpha Mating Factor Pre-Sequence
EFETMRFPSIFTAVLFAASSALA
Human Serum Albumin
SEQ ID NO: 40
MKWVTFISLLFLESSAYS
Bovine DNASE1
SEQ ID NO: 41
MRGTRLMGLLLALAGLLOLGLS
Bovine DNASE1 + Kex2-Site
SEQ ID NO: 42
MRGTRLMGLLLALAGLLQLGLSLEKR
Alpha Amylase
SEQ ID NO: 43
EFETMRFPSI FTAVLFAASSALA
Glucoamylase Signal Peptide
SEQ ID NO: 44
EFETMSFRSLLALSGLVCSGLA
Inulinase
SEQ ID NO: 45
EFETMKLAYSLLLPLAGVSA
Invertase
SEQ ID NO: 46
EFETMLLQAFLFLLAGFAAKISA
Killer protein
SEQ ID NO: 47
EFETMTKPTQVLVRSVSILFFITLLHLVVA
Lysozyme
SEQ ID NO: 48
EFETMLGKNDPMCLVLVLLGLTALLGICQG
A synthetic E. coli signal peptide
SEQ ID NO: 49
MKKNIAFLLALMEVESIATNAYA
A synthetic E. coli signal peptide
SEQ ID NO: 50
MKKNIAFLLAIMFVESIATNAYA
Salmonella enterica DsbA signal peptide
SEQ ID NO: 51
MKKIWLALAGIVLAFSASA
A synthetic E. coli signal peptide
SEQ ID NO: 52
MKKIWLALAGLVLAFSAYA
A synthetic E. coli signal peptide
SEQ ID NO: 53
MKKNIAFLLAAMFVESIATNAYA
A synthetic E. coli signal peptide
SEQ ID NO: 54
MKKNILFLLLLMFVESIATNAYA
E. coli FimD signal peptide
SEQ ID NO: 55
MMTKIKLLMLIIFYLIISASAHA
E. coli OmpA signal peptide
SEQ ID NO: 56
MKKRARAIAIAVALAGFATVAHA
A signal peptide from Bordetella pertussis
SEQ ID NO: 57
MKKWFVAAGIGAGLLMLSSAA
E. coli DsbA signal peptide
SEQ ID NO: 67
KKIWLALAGLVLAFSASA
E. coli ST-II signal peptide
SEQ ID NO: 68
MKKNIAFLLASMFVESIATNAYA

### LINKERS SEPARATING SIGNAL PEPTIDES FROM SECRETED PROTEINS

SEQ ID NO: 58
GGGGS
SEQ ID NO: 59
GGGGSGGGGSGGGGS
SEQ ID NO: 60
SGGGG
SEQ ID NO: 74
CGGGG
SEQ ID NO: 61
SGGSGGSGG
SEQ ID NO: 62
SGGSGGSGGSGGSGGSGG
SEQ ID NO: 69
SGGSGGSGSS
C-terminal Basic Domain
SEQ ID NO: 75
SSRAFTNSKKSVTLRKKTKSKRS

What is claimed is:

1. A variant of DNASE1-LIKE 3 (D1L3 variant) comprising an N-terminal extension of at least 4 amino acids and no more than about 18 amino acids relative to SEQ ID NO: 4.

2. The D1L3 variant of claim 1, wherein the N-terminal amino acid is not Met.

3. The D1L3 variant of claim 1 or 2, wherein the N-terminal extension comprises predominately Gly residues.

4. The D1L3 variant of any one of claims 1 to 3, wherein the DIL3 variant comprises an amino acid sequence that has at least 80%, or at least 85%, or at least 90%, or at least 95%, or at least 97% %, or at least 98%, or at least 99% sequence identity to amino acids 21 to 282 of SEQ ID NO: 4 or amino acids 21 to 252 of SEQ ID NO: 5.

5. The D1L3 variant of any one of claims 1 to 4, wherein the D1L3 variant is produced by cleavage of an N-terminal signal peptide selected from SEQ ID NO: 37, SEQ ID NO: 38, SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, and SEQ ID NO: 48.

6. The D1L3 variant of claim 5, wherein the signal peptide is the alpha-mating factor (αMF) pre-pro secretion leader from Saccharomyces cerevisiae (SEQ ID NO: 38).

7. The D1L3 variant of any one of claims 1 to 6, wherein the N-terminal extension is at least four, or at least five, or at least six, or at least seven, or at least eight, or at least nine, or at least ten, or at least 11, or at least 12, or at least 13, or at least 14, or at least 15 amino acids in length.

8. The DIL3 variant of claim 7, wherein the N-terminal extension consists essentially of Ser and Gly residues, or consists of Ser and Gly residues.

9. The D1L3 variant of claim 8, wherein the N-terminal extension and or the junction of the N-terminal extension and sequence originating from D1L3 is non-immunogenic.

10. The D1L3 variant of any one of claims 7 to 9, wherein the N-terminal extension comprises an amino acid having a chemical group suitable for chemical conjugation, the chemical group being selected from a thiol group, amino group, amido group, and carboxyl group.

11. The D1L3 variant of any one of claims 7 to 10, wherein the N-terminal extension has an amino acid sequence of SGGGG (SEQ ID NO: 60) or CGGGG (SEQ ID NO: 74).

12. The D1L3 variant of any one of claims 1 to 11, wherein the N-terminal extension does not include a consensus sequence for myristoylation.

13. The D1L3 variant of any one of claims 1 to 12, wherein the DIL3 variant further comprises a C-terminal extension reducing heterogeneity.

14. The D1L3 variant of claim 13, wherein the C-terminal extension is at least two, or at least three, or at least four, or at least five amino acids in length.

15. The D1L3 variant of any one of claims 1 to 14, wherein the C-terminal amino acid is Lys or Arg.

16. The D1L3 variant of any one of claims 13 to 15, wherein the C-terminal extension comprises or consists of the amino acid sequence SSR.

17. The D1L3 variant of any one of claims 1 to 16, wherein the DIL3 variant comprises the basic domain.

18. The DIL3 variant of any one of claims 1 to 17, wherein the D1L3 variant comprises a deletion of at least three, or at least five, or at least eight, or at least ten, or at least twelve, or at least fifteen, or at least eighteen, or at least twenty, or all 23 amino acids of a C-terminal basic domain (BD) defined by amino acids 283 to 305 of SEQ ID NO: 4.

19. The D1L3 variant of any one of claims 1 to 18, wherein the DIL3 variant is configured to form a dimer via an unpaired Cys, wherein the unpaired Cys is optionally C48 with respect to SEQ ID NO: 4.

20. The D1L3 variant of any one of claims 1 to 18, wherein the D1L3 variant comprises a substitution of C48 and/or C174 with respect to SEQ ID NO: 4.

21. The D1L3 variant of claim 20, wherein the mutation is selected from C48S, C48G, C48A, C174S, C174G and C174A with respect to SEQ ID NO: 4.

22. The D1L3 variant of claim 21, wherein the substitution is C48A with respect to SEQ ID NO: 4.

23. The DIL3 variant of any one of claims 1 to 22, wherein the C-terminal extension comprises or consists of the amino acid sequence SSR.

24. The D1L3 variant of any one of claims 1 to 23, wherein the DIL3 variant comprises one or more mutations that result in resistance to proteolysis by one or more of plasmin, thrombin, trypsin, and proteases produced by mammalian and non-mammalian cell lines.

25. The D1L3 variant of claim 24, wherein the DIL3 variant has one or more mutations of amino acid residues selected from K180, K200, K259, and R285 with respect to SEQ ID NO: 4.

26. The D1L3 variant of claim 24 or claim 25, wherein the DIL3 variant has one or more mutations of amino acid residues selected from R22, R29, K45, K47, K74, R81, R92, K107, K176, R212, R226, R227, K250, K259, and K262 with respect to SEQ ID NO: 4.

27. The D1L3 variant of claim 25 or claim 26, wherein the DIL3 variant has one or more mutations of amino acid residues selected from S91C, S131C, and S253C with respect to SEQ ID NO: 4.

28. The D1L3 variant of claim 1, having the amino acid sequence of SEQ ID NO: 63, SEQ ID NO: 66, SEQ ID NO: 70, SEQ ID NO: 71, SEQ ID NO: 72, SEQ ID NO: 73, or SEQ ID NO: 77.

29. The D1L3 variant of claim 28, having the amino acid sequence of SEQ ID NO: 70.

30. The D1L3 variant of any one of claims 1 to 29, wherein the DIL3 variant comprises a fusion or conjugation to a half-life extending moiety.

31. The DIL3 variant of claim 30, wherein the half-life extending moiety is a polymer.

32. The D1L3 variant of claim 31, wherein the polymer is a polyethylene glycol (PEG).

33. The D1L3 variant of claim 32, wherein a PEG polymer is conjugated to the N-terminus.

34. The D1L3 variant of claim 33, wherein the PEG polymer connects two DIL3 variant molecules through their N-termini.

35. The D1L3 variant of any one of claims 31-33, wherein a PEG polymer is conjugated to one or more amino acids within positions corresponding to R95 to V126 of SEQ ID NO: 4.

36. The D1L3 variant of claim 35, wherein one or more PEGylated amino acids are selected from lysine, cysteine, histidine, arginine, aspartic acid, glutamic acid, serine, threonine and tyrosine, optionally wherein one or more PEGylated amino acids are introduced by substitution of one or more amino acids between R95 and V126 relative to SEQ ID NO: 4.

37. The D1L3 variant of claim 36, wherein the one or more amino acids are PEGylated by:
(a) PEGylation of lysine (Lys or K) conducted via amine conjugation;
(b) PEGylation of glutamine conducted via transglutaminase (TGase) mediated enzymatic conjugation; and/or
(c) PEGylation of cysteine (Cys or C) conducted via thiol conjugation.

38. The DIL3 variant of any one of claims 32 to 37, wherein one or more PEGylated amino acids are conjugated with PEG moieties that are independently selected from a linear or branched PEG having molecular weights that are independently selected and in the range of about 2 kDa to about 60 kDa, or about 5 kDa to about 30 kDa.

39. The D1L3 variant of claim 30, wherein the half-life extending moiety is a fusion partner.

40. The D1L3 variant of claim 39, wherein the fusion partner is selected from albumin, transferrin, an Fc, or elastin-like protein, XTEN sequence, or a variant thereof.

41. The D1L3 variant of claim 40, wherein the fusion partner is an albumin.

42. The D1L3 variant of claim 41, wherein the fusion partner is a human albumin comprising an amino acid sequence that is at least 80% identical to SEQ ID NO: 26.

43. The DIL3 variant of claim 42, wherein the human albumin comprises E505Q, T527M, K573P substitutions with respect to SEQ ID NO: 26.

44. The D1L3 variant of any one of claims 39 to 43, wherein the fusion partner is fused at the N-terminus to mature D1L3 enzyme.

45. The D1L3 variant of any one of claims 39 to 44, wherein the fusion partner is fused to the mature D1L3 enzyme via a linker adjoining the fusion partner and the mature D1L3 enzyme.

46. The D1L3 variant of claim 45, wherein the linker is from about 5 to about 50 amino acids, or from about 10 to about 35 amino acids, or from about 15 to about 35 amino acids in length.

47. The D1L3 variant of claim 46, wherein the linker comprises the amino acid sequence S(GGS)₄GSS (SEQ ID NO: 23), S(GGS)₉GSS (SEQ ID NO: 24), and (GGS)₉GS (SEQ ID NO: 25).

48. The D1L3 variant of claim 46 or 47, wherein the DIL3 variant comprises the amino acid sequence of SEQ ID NO: 76 or SEQ ID NO: 78.

49. The D1L3 variant of claim 45 or 46, wherein the linker is a flexible or rigid linker, and/or comprises a protease cleavage site.

50. The D1L3 variant of claim 49, wherein the linker is cleavable by a coagulation pathway protease.

51. The D1L3 variant of claim 50, wherein the protease is Factor XII or a neutrophil protease.

52. The DIL3 variant of claim 51, wherein the protease is thrombin.

53. The D1L3 variant of claim 50 or 51, wherein the protease cleavage site comprises the amino acid sequence LVPRG (SEQ ID NO: 64), and optionally SGGGGLVPRGSGGGG (SEQ ID NO: 65).

54. A method for expressing the DNase1-like 3 variant of any one of claims 1 to 53, comprising:
introducing a genetic construct encoding the DNase1-like 3 variant comprising a signal peptide in a yeast cell, and
recovering the DNase1-like 3 variant.

55. The method of claim 54, wherein the yeast cell is Pichia pastoris.

56. The method of claim 54 or claim 55, wherein the signal peptide is the signal peptide is the alpha-mating factor (αMF) pre-pro secretion leader from Saccharomyces cerevisiae (SEQ ID NO: 38).

57. An isolated polynucleotide encoding the DIL3 variant of any one of claims 1 to 53.

58. The isolated polynucleotide of claim 57, wherein the polynucleotide is an mRNA or a modified mRNA (mmRNA).

59. The isolated polynucleotide of claim 58, wherein the polynucleotide is DNA.

60. A vector for introducing the polynucleotide of any one of claims 57 to 59 to a host cell.

61. A host cell comprising the vector of claim 60.

62. A pharmaceutical composition comprising an effective amount of the DIL3 variant of any one of claims 1 to 53, or the D1L3 variant produced according the method of any one of claims 54 to 56, or the polynucleotide according to any one of claims 57 to 59, or the vector according to claim 60, or the host cell according to claim 61, and a pharmaceutically acceptable carrier.

63. The pharmaceutical composition of claim 62, wherein the composition comprises the D1L3 variant of any one of claims 1 to 53, and a pharmaceutically acceptable carrier for parenteral administration.

64. The pharmaceutical composition of claim 62, formulated for ocular or pulmonary administration.

65. The pharmaceutical composition of claim 63, formulated for intradermal, intramuscular, intravenous, subcutaneous, or intraarterial administration.

66. A method for treating a subject in need of extracellular chromatin degradation, the method comprising administering the pharmaceutical composition of any one of claims 63 to 65 to a subject in need thereof.

67. The method of claim 66, wherein the subject is in need of extracellular trap (ET) degradation and/or neutrophil extracellular trap (NET) degradation.

68. The method of claim 66 or 67, wherein the subject has a loss of function mutation in one or both DIL3 genes.

69. The method of any one of claim 67 or 68, wherein the subject has SLE.

70. The method of any one of claims 66 to 69, wherein the subject has a condition selected from chronic neutrophilia, neutrophil aggregation or leukostasis, thrombosis or vascular occlusion, ischemia-reperfusion injury, surgical or traumatic tissue injury, an acute or chronic inflammatory reaction or disease, an autoimmune disease, cardiovascular disease, metabolic disease, systemic inflammation, inflammatory disease of the respiratory tract, renal inflammatory disease, inflammatory disease related to transplanted tissue and cancer.

71. The method of any one of claims 66 to 69, wherein the subject has, or is at risk of, NETs occluding ductal systems, wherein the condition is optionally selected from pancreatitis, cholangitis, conjunctivitis, mastitis, dry eye disease, obstructions of vas deferens, and renal disease.

72. The method of any one of claims 66 to 71, wherein the subject has, or is at risk of, NETs accumulating on endothelial surfaces.

73. An expression construct for improving processing of a polypeptide precursor in a host, the expression construct comprising a signal peptide fused to the polypeptide via a linker of at least three amino acids length.

74. The expression construct of claim 73, wherein the signal peptide is incompletely processed in the host in the absence of the linker.

75. The expression construct of claim 73 or claim 74, wherein the polypeptide is selected from an enzyme, a cytokine, a hormone, an antibody or an antigen binding fragment thereof, an antibody-like molecule or an antigen binding fragment thereof, antigen, a component of a vaccine, a fusion protein and a combination thereof.

76. The expression construct of claim 75, wherein the enzyme is selected from DNASE1 (D1), DNASE1-LIKE 1 (DIL1), DNASE1-LIKE 2 (DIL2), DNASE1-LIKE 3 Isoform 1 (DIL3), DNASE1-LIKE 3 Isoform 2 (DIL3-2), DNASE2A (D2A), and DNASE2B (D2B), or a variant thereof.

77. The expression construct of any one of claims 73 to 76, wherein the linker is at least 5 amino acids in length, or is at least 9 amino acids in length, or is at least 12 amino acids in length, and is predominately serine and glycine residues.

78. The expression construct of any one of claims 73 to 77, wherein the N-terminal extension and or the junction of the N-terminal extension and sequence originating from D1L3 is non-immunogenic.

79. The expression construct of any one of claims 73 to 78, wherein the N-terminal extension comprises an amino acid having a chemical group suitable for chemical conjugation, the chemical group being selected from a thiol group, amino group, amido group, and carboxyl group.

80. The expression construct of any one of claims 73 to 79, wherein the signal peptide is selected from DNASE1L3 (SEQ ID NO: 37), Alpha Mating Factor (SEQ ID NO: 38), Alpha Mating Factor Pre-Sequence (SEQ ID NO: 39), Human Serum Albumin (SEQ ID NO: 40), Bovine DNASE1 (SEQ ID NO: 41), Bovine DNASE1+Kex2-Site (SEQ ID NO: 42), Alpha Amylase (SEQ ID NO: 43), Glucoamylase Signal Peptide (SEQ ID NO: 44), Inulinase (SEQ ID NO: 45), Invertase (SEQ ID NO: 46), Killer protein (SEQ ID NO: 47), and Lysozyme (SEQ ID NO: 48).

81. The expression construct of any one of claims 73 to 80, wherein the host is a yeast, or a cell line selected from a mammalian cell line, and insect cell line.

82. The expression construct of claim 81, wherein the host is Pichia pastoris.

83. The expression construct of claim 82, wherein the signal peptide is alpha-mating factor (αMF) pre-pro secretion leader from Saccharomyces cerevisiae (SEQ ID NO: 38).


SEQUENCE LISTING (81 sequences)

SEQ ID NO: 1 - protein, 282 aa, synthetic construct [sequence omitted]

SEQ ID NO: 2 - protein, 302 aa, synthetic construct
MHYPTALLFLILANGAQAFRICAFNAQRLTLAKVAREQVMDTLVRILARCDIMVLQEVVDSSGSAIPLLLRELNRFDGSGPYSTLSSPQLGRSTYMETYVYFYRSHKTQVLSSYVYNDEDDVFAREPFVAQFSLPSNVLPSLVLVPLHTTPKAVEKELNALYDVFLEVSQHWQSKDVILLGDFNADCASLTKKRLDKLELRTEPGFHWVIADGEDTTVRASTHCTYDRVVLHGERCRSLLHTAAAFDFPTSFQLTEEEALNISDHYPVEVELKLSQAHSVQPLSLTVLLLLSLLSPQLCPAA

SEQ ID NO: 3 - protein, 299 aa, synthetic construct
MGGPRALLAALWALEAAGTAALRIGAFNIQSFGDSKVSDPACGSIIAKILAGYDLALVQEVRDPDLSAVSALMEQINSVSEHEYSFVSSQPLGRDQYKEMYLFVYRKDAVSVVDTYLYPDPEDVFSREPFVVKFSAPGTGERAPPLPSRRALTPPPLPAAAQNLVLIPLHAAPHQAVAEIDALYDVYLDVIDKWGTDDMLFLGDFNADCSYVRAQDWAAIRLRSSEVFKWLIPDSADTTVGNSDCAYDRIVACGARLRRSLKPQSATVHDFQEEFGLDQTQALAISDHFPVEVTLKFHR

SEQ ID NO: 4 - protein, 305 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 5 - protein, 275 aa, synthetic construct
MSRELAPLLLLLLSIHSALAMRICSFNVRSFGESKQEDKNAMDVIVKVIKRCDIILVMEIKDSNNRICPILMEKLNREKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 6 - protein, 360 aa, synthetic construct
MIPLLLAALLCVPAGALTCYGDSGQPVDWFVVYKLPALRGSGEAAQRGLQYKYLDESSGGWRDGRALINSPEGAVGRSLQPLYRSNTSQLAFLLYNDQPPQPSKAQDSSMRGHTKGVLLLDHDGGFWLVHSVPNFPPPASSAAYSWPHSACTYGQTLLCVSFPFAQFSKMGKQLTYTYPWVYNYQLEGIFAQEFPDLENVVKGHHVSQEPWNSSITLTSQAGAVFQSFAKFSKFGDDLYSGWLAAALGTNLQVQFWHKTVGILPSNCSDIWQVLNVNQIAFPGPAGPSFNSTEDHSKWCVSPKGPWTCVGDMNRNQGEEQRGGGTLCAQLPALWKAFQPLVKNYQPCNGMARKPSRAYKI

SEQ ID NO: 7 - protein, 361 aa, synthetic construct
MKQKMMARLLRTSFALLFLGLFGVLGAATISCRNEEGKAVDWFTFYKLPKRQNKESGETGLEYLYLDSTTRSWRKSEQLMNDTKSVLGRTLQQLYEAYASKSNNTAYLIYNDGVPKPVNYSRKYGHTKGLLLWNRVQGFWLIHSIPQFPPIPEEGYDYPPTGRRNGQSGICITFKYNQYEAIDSQLLVCNPNVYSCSIPATFHQELIHMPQLCTRASSSEIPGRLLTTLQSAQGQKFLHFAKSDSFLDDIFAAWMAQRLKTHLLTETWQRKRQELPSNCSLPYHVYNIKAIKLSRHSYFSSYQDHAKWCISQKGTKNRWTCIGDLNRSPHQAFRSGGFICTQNWQIYQAFQGLVLYYESCK

SEQ ID NO: 8 - protein, 304 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKR

SEQ ID NO: 9 - protein, 302 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKS

SEQ ID NO: 10 - protein, 293 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKS

SEQ ID NO: 11 - protein, 290 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNS

SEQ ID NO: 12 - protein, 284 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSS

SEQ ID NO: 13 - protein, 282 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQ

SEQ ID NO: 14 - protein, 297 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLR

SEQ ID NO: 15 - protein, 296 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTL

SEQ ID NO: 16 - protein, 292 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKK

SEQ ID NO: 17 - protein, 291 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSK

SEQ ID NO: 18 - protein, 5 aa, synthetic construct
GGGGS

SEQ ID NO: 19 - protein, 15 aa, synthetic construct
GGGGSGGGGSGGGGS

SEQ ID NO: 20 - protein, 14 aa, synthetic construct
APAPAPAPAPAPAP

SEQ ID NO: 21 - protein, 12 aa, synthetic construct
AEAAAKEAAAKA

SEQ ID NO: 22 - protein, 7 aa, synthetic construct
SGGSGSS

SEQ ID NO: 23 - protein, 16 aa, synthetic construct
SGGSGGSGGSGGSGSS

SEQ ID NO: 24 - protein, 31 aa, synthetic construct
SGGSGGSGGSGGSGGSGGSGGSGGSGGSGSS

SEQ ID NO: 25 - protein, 29 aa, synthetic construct
GGSGGSGGSGGSGGSGGSGGSGGSGGSGS

SEQ ID NO: 26 - protein, 585 aa, synthetic construct [sequence omitted]

SEQ ID NO: 27 - protein, 625 aa, synthetic construct
MIFLYQVVHFILFTSVSGECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 28 - protein, 638 aa, synthetic construct
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA

SEQ ID NO: 29 - protein, 25 aa, synthetic construct
CTTKIKPRIVGGTASVRGEWPWQVT

SEQ ID NO: 30 - protein, 13 aa, synthetic construct
GGGGSPRIGGGGS

SEQ ID NO: 31 - protein, 26 aa, synthetic construct
VCTTKTSTRIVGGTNSSWGEWPWQVS

SEQ ID NO: 32 - protein, 7 aa, synthetic construct
STRIVGG

SEQ ID NO: 33 - protein, 878 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGSGGSGGSGGSGGSGGSGGSGGSGGSGSSMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQ

SEQ ID NO: 34 - protein, 538 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSGGSGGSGGSGGSGGSGGSGGSGGSGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSVSVMHEALHNHYTQKSLSLSPGK

SEQ ID NO: 35 - protein, 885 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 36 - protein, 560 aa, synthetic construct
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSSGGSGGSGGSGGSGGSGGSGGSGGSGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSVSVMHEALHNHYTQKSLSLSPGK

SEQ ID NO: 37 - protein, 20 aa, synthetic construct
MSRELAPLLLLLLSIHSALA

SEQ ID NO: 38 - protein, 85 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKR

SEQ ID NO: 39 - protein, 23 aa, synthetic construct
EFETMRFPSIFTAVLFAASSALA

SEQ ID NO: 40 - protein, 18 aa, synthetic construct
MKWVTFISLLFLFSSAYS

SEQ ID NO: 41 - protein, 22 aa, synthetic construct
MRGTRLMGLLLALAGLLQLGLS

SEQ ID NO: 42 - protein, 26 aa, synthetic construct
MRGTRLMGLLLALAGLLQLGLSLEKR

SEQ ID NO: 43 - protein, 23 aa, synthetic construct
EFETMRFPSIFTAVLFAASSALA

SEQ ID NO: 44 - protein, 22 aa, synthetic construct
EFETMSFRSLLALSGLVCSGLA

SEQ ID NO: 45 - protein, 20 aa, synthetic construct
EFETMKLAYSLLLPLAGVSA

SEQ ID NO: 46 - protein, 23 aa, synthetic construct
EFETMLLQAFLFLLAGFAAKISA

SEQ ID NO: 47 - protein, 30 aa, synthetic construct
EFETMTKPTQVLVRSVSILFFITLLHLVVA

SEQ ID NO: 48 - protein, 30 aa, synthetic construct
EFETMLGKNDPMCLVLVLLGLTALLGICQG

SEQ ID NO: 49 - protein, 23 aa, synthetic construct
MKKNIAFLLALMFVFSIATNAYA

SEQ ID NO: 50 - protein, 23 aa, synthetic construct
MKKNIAFLLAIMFVFSIATNAYA

SEQ ID NO: 51 - protein, 19 aa, synthetic construct
MKKIWLALAGIVLAFSASA

SEQ ID NO: 52 - protein, 19 aa, synthetic construct
MKKIWLALAGLVLAFSAYA

SEQ ID NO: 53 - protein, 23 aa, synthetic construct
MKKNIAFLLAAMFVFSIATNAYA

SEQ ID NO: 54 - protein, 23 aa, synthetic construct
MKKNILFLLLLMFVFSIATNAYA

SEQ ID NO: 55 - protein, 23 aa, synthetic construct
MMTKIKLLMLIIFYLIISASAHA

SEQ ID NO: 56 - protein, 23 aa, synthetic construct
MKKRARAIAIAVALAGFATVAHA

SEQ ID NO: 57 - protein, 21 aa, synthetic construct
MKKWFVAAGIGAGLLMLSSAA

SEQ ID NO: 58 - protein, 5 aa, synthetic construct
GGGGS

SEQ ID NO: 59 - protein, 15 aa, synthetic construct
GGGGSGGGGSGGGGS

SEQ ID NO: 60 - protein, 5 aa, synthetic construct
SGGGG

SEQ ID NO: 61 - protein, 9 aa, synthetic construct
SGGSGGSGG

SEQ ID NO: 62 - protein, 18 aa, synthetic construct
SGGSGGSGGSGGSGGSGG

SEQ ID NO: 63 - protein, 352 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRSGGGGMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQ

SEQ ID NO: 64 - protein, 5 aa, synthetic construct
LVPRG

SEQ ID NO: 65 - protein, 15 aa, synthetic construct
SGGGGLVPRGSGGGG

SEQ ID NO: 66 - protein, 355 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRSGGGGMRICSFNVRSFGESKQEDKNAMDVIVKVIKRCDIILVMEIKDSNNRISPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSR

SEQ ID NO: 67 - protein, 18 aa, synthetic construct
KKIWLALAGLVLAFSASA

SEQ ID NO: 68 - protein, 23 aa, synthetic construct
MKKNIAFLLASMFVFSIATNAYA

SEQ ID NO: 69 - protein, 10 aa, synthetic construct
SGGSGGSGSS

SEQ ID NO: 70 - protein, 355 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRSGGGGMRICSFNVRSFGESKQEDKNAMDVIVKVIKRCDIILVMEIKDSNNRIAPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSR

SEQ ID NO: 71 - protein, 355 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRCGGGGMRICSFNVRSFGESKQEDKNAMDVIVKVIKRCDIILVMEIKDSNNRIAPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSR

SEQ ID NO: 72 - protein, 355 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRCGGGGMRICSFNVRSFGESKQEDKNAMDVIVKVIKRCDIILVMEIKDSNNRISPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSR

SEQ ID NO: 73 - protein, 375 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKRCGGGGMRICSFNVRSFGESKQEDKNAMDVIVKVIKRCDIILVMEIKDSNNRIAPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 74 - protein, 5 aa, synthetic construct
CGGGG

SEQ ID NO: 75 - protein, 23 aa, synthetic construct
SSRAFTNSKKSVTLRKKTKSKRS

SEQ ID NO: 76 - protein, 879 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGGSGGGGSGGGGSGGGGMRICSFNVRSFGESKQEDKNAMDVIVKVIKRCDIILVMEIKDSNNRIAPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSR

SEQ ID NO: 77 - protein, 270 aa, synthetic construct
SGGGGMRICSFNVRSFGESKQEDKNAMDVIVKVIKRCDIILVMEIKDSNNRIAPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSR

SEQ ID NO: 78 - protein, 880 aa, synthetic construct
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLSGGSGGSGGSGGSGGSGGSGGSGGSGGSGGMRICSFNVRSFGESKQEDKNAMDVIVKVIKRCDIILVMEIKDSNNRIAPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSR

SEQ ID NO: 79 - protein, 20 aa, synthetic construct
SGGSGGSGSSMRICSFNVRS

SEQ ID NO: 80 - protein, 15 aa, synthetic construct
SGGGGMRICSFNVRS

SEQ ID NO: 81 - protein, 19 aa, synthetic construct
SGGSGGSGGMRICSFNVRS